(19) United States
(12) Patent Application Publication
(10) Pub. No.: US 2025/0017737 A1
(43) Pub. Date: Jan. 16, 2025

(54) REVERSE SHOULDER PROSTHESIS AND RELATED METHODS

(71) Applicant: Skeletal Dynamics, Inc., Miami, FL (US)

(72) Inventors: Jorge L. ORBAY, Miami, FL (US); Robert SIXTO, Miami, FL (US); Francisco RUBIO, Miami, FL (US); Juergen Kortenbach, Miami, FL (US)

(21) Appl. No.: 18/830,152

(22) Filed: Sep. 10, 2024

Related U.S. Application Data
(63) Continuation-in-part of application No. 17/649,142, filed on Jan. 27, 2022.
(60) Provisional application No. 63/581,796, filed on Sep. 11, 2023.
(60) Provisional application No. 63/142,344, filed on Jan. 27, 2021.

(51) Int. Cl. A61F 2/40; A61F 2/30

(52) U.S. Cl. CPC A61F 2/4014; A61F 2002/30649; A61F 2002/4022; A61F 2002/4051; A61F 2002/4062; A61F 2002/4085

(57) ABSTRACT

Disclosed is a prosthetic joint assembly and method for joining a humerus bone to scapula bone, comprising a humeral component for engagement with said humerus bone and having a concave dish; a scapular component for engagement with said scapula bone and having a convex surface adapted to engage said concave dish; wherein the geometry of said components when implanted and engaged is such that the humerus bone is relocated with respect to the scapula bone by calculating a humeral reference point medial to a natural center of rotation (COR), and a scapular reference point approximately normal and medial to the humeral reference point, relocating the humerus bone so the humeral reference point superimposes the scapular reference point, establishing a prosthetic COR at the scapular reference point. Also disclosed is a scapular baseplate having a first side with a mating surface and scapular stem, and a second side having a trunnion.

### CLAIM OF PRIORITY

This application is a continuation-in-part of U.S. non-provisional patent application Ser. No. 17/649,142, filed on Jan. 27, 2022, which claims priority under 35 U.S.C. § 119 (e) to U.S. provisional patent application Ser. No. 63/142,344 filed on Jan. 27, 2021. This application also claims priority under 35 U.S.C. § 119 (e) to U.S. provisional patent application Ser. No. 63/581,796, filed on Sep. 11, 2023. The contents of all of the above are incorporated herein by reference.

### FIELD OF INVENTION

The invention relates generally to prosthetic implants and in particular to prosthetic implants for use in a total shoulder replacement procedure.

### BACKGROUND OF THE INVENTION

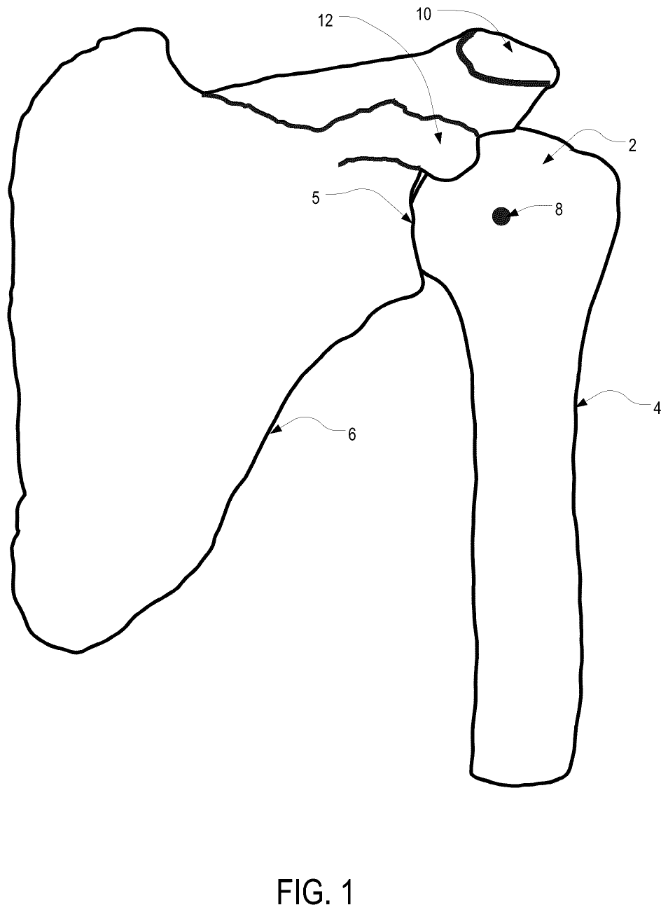

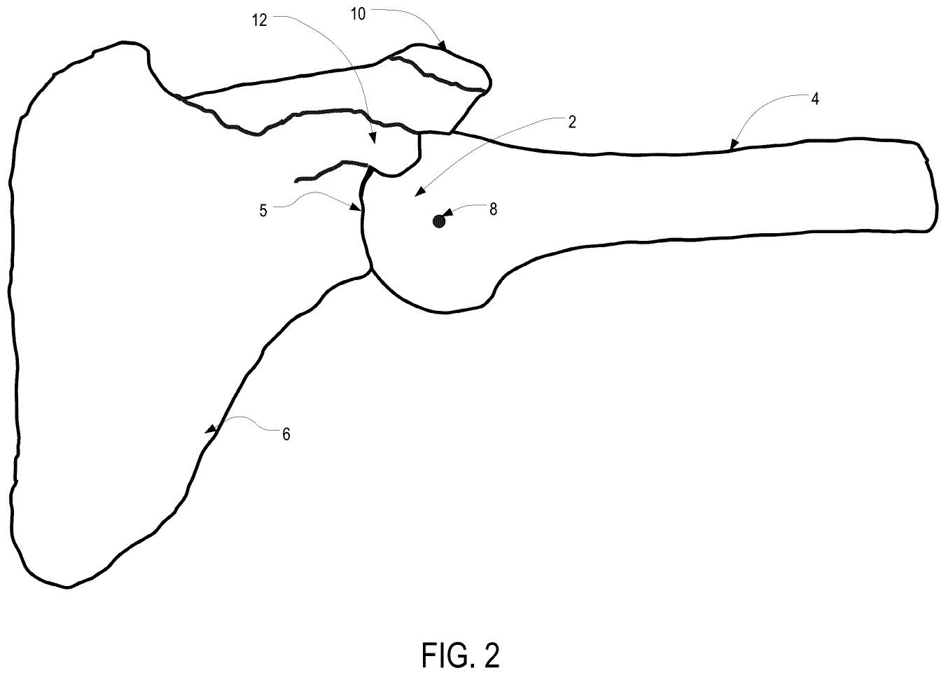

FIGS. 1 and 2 illustrate the features of the normal human shoulder in the rest and abducted position respectively. The human shoulder joint is formed where the head (2) of the humerus (upper arm) bone (4) engages the glenoid cavity (5) of the scapula (shoulder blade) bone (6). In normal functioning of a healthy shoulder, the articular surface of the humeral head (2) fits into the glenoid cavity (5) like a ball and socket, allowing the humerus (4) to freely swivel with respect to the scapula (6) while being retained within the shoulder joint. This swiveling motion occurs about a center of rotation (8) (hereinafter “natural center of rotation” or “nCOR”) between the humerus bone (4) and the scapula bone (6) which is usually located at or near the center of the humeral head (2). The nCOR as referenced herein is for a healthy joint. In a healthy shoulder the upper movement of the humerus (4) (on the frontal plane) is bound by a protrusion of the scapula (6) called the acromion (10), and in particular by a feature of the scapula called the coracoid process (12). Furthermore, in a healthy shoulder the humeral head (2) is retained within the glenoid cavity (5) by a complex of muscles and tendons commonly referred to as the rotator cuff (not shown for clarity) which surround and stabilize the shoulder joint.

Due to injury, trauma, degenerative changes, disease (such as arthritis) or other conditions, a person may experience pain, discomfort, or difficulty when operating the shoulder through its range of motion or may not be able to operate the shoulder at all. In certain situations, shoulder joint conditions may be addressed through a partial replacement of the joint. In a partial replacement, the head (2) of the humerus (4) is replaced by a prosthetic implant, while the glenoid cavity (5) is left relatively intact. In many cases, however, because the glenoid cavity (5) is too deteriorated or damaged to engage and hold the head (2) of the humerus (4), a partial replacement is not advised or possible.

In such cases, where a partial replacement is not possible, one available treatment is to replace the head (2) of the humerus (4) as well as the glenoid cavity (5) with a prosthetic shoulder in a procedure commonly referred to as a total shoulder replacement. Moreover, because in most situations where a total shoulder replacement is required the rotator cuff is also damaged and unable to stabilize the head (2) of the humerus (4) within the glenoid cavity (5), the configuration of the components in the total shoulder prosthesis is reversed. That is, in a reverse total shoulder prosthesis, the component implanted on the scapula, (corresponding to the glenoid cavity (5)) is convex, or ball-shaped, while the component implanted on the head (2) of the humerus (4) is concave, or socket-shaped. Such reverse configuration has been found to be more stable in the absence of a fully healthy rotator cuff.

Although previous efforts have been made to develop reverse total shoulder prostheses, they have met often with disappointing results. Presently available reverse total shoulder prostheses provide too limited a range of motion, dislocate too easily, place too much stress on bones resulting in failure of the prostheses, bone fractures, or both, cause complications such as infections, and wear prematurely requiring additional surgeries during the patient's lifetime, among other flaws. In addition, the methods presently used for implanting such prosthetic shoulder joints often result in poorly aligned joints and poor joint performance and range of movement.

Accordingly, there is a need in the art for a reverse total shoulder prosthesis, and associated methods for implanting same, which provides a patient with a range of motion and alignment that approximates that of a healthy shoulder, is long-lasting, provides adequate support for the remaining upper arm and chest bones, and avoids some or all of the drawbacks of existing prosthetic shoulders.

### SUMMARY OF THE INVENTION

It has been determined by the inventors herein that the deficiencies in existing total reverse shoulder prostheses are primarily due to (a) the improper location of the center of rotation between the prosthetic scapular and humeral components (hereinafter the “prosthetic center of rotation” or “pCOR”); and (b) improper absolute placement of the humeral bone relative to the scapula once the prosthesis is in place.

The present invention provides a novel reverse total shoulder prosthesis that once implanted properly places the pCOR and humeral bone in order to provide an optimally functioning prosthetic shoulder joint. More specifically, the pCOR is placed in a position that is medial and inferior to the position of the nCOR. Additionally, for optimal placement, the humerus is translated in a direction that is inferior to the location of the nCOR.

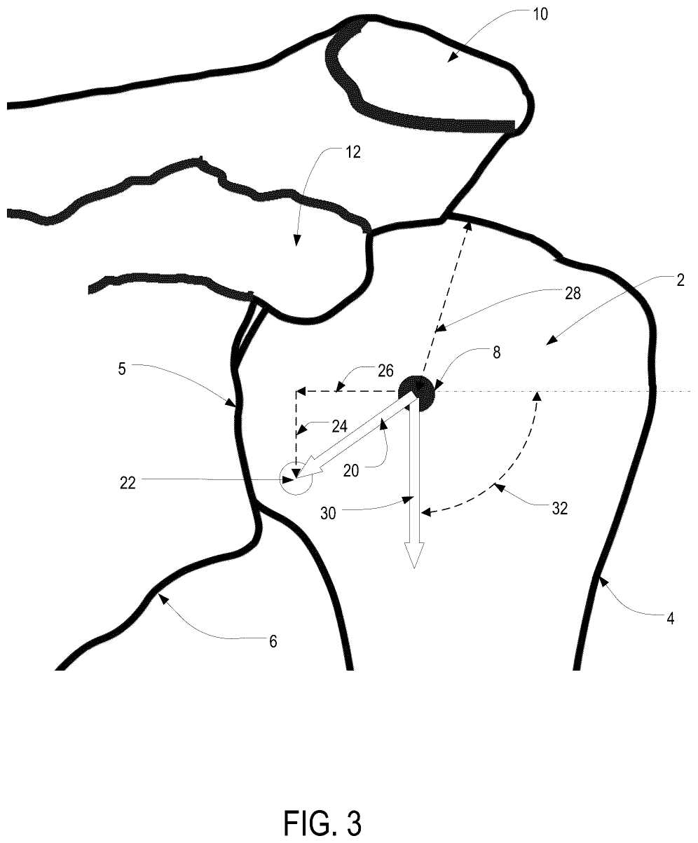

Referring to FIG. 3, which shows a close-up view of the interface between the scapula (6) and humerus bone (4), the inventors have determined through experimentation and simulation that the vector (20) of translation of the natural center of rotation (8) to the optimal pCOR location (22) includes a range of ratios between the inferior (24) and medial (26) components of that vector (20). That range of ratios between the inferior (24) and medial (26) components of the vector of translation of the center of rotation (20) is between 0.6 and 1.2 (resulting in an angle range of between 30 and 50 degrees below horizontal), with a preferred ratio being between 0.85 and 1.15 (resulting in an angle range of between 40 and 49 degrees below horizontal). The optimal solution in most cases is one where the medial (26) and inferior (24) components are equal, or have a ratio of 1 (resulting in an angle of 45 degrees below horizontal). The optimal magnitude of the vector of translation of the center of rotation (20) similarly has a range, which is between 60% and 80% of the radius (28) of the humeral head (2) of the patient. In most cases the optimal magnitude of the vector of translation of the center of rotation (20) is about 70% of the radius (28) of the humeral head (2) of the patient.

The inventors have similarly determined that the vector of translation of the humerus (30) in the inferior direction with respect to the nCOR (8) has a direction angle (32) between 75 and 105 degrees below horizontal. In most cases, the optimal solution occurs where the vector of translation of the humerus (30) is 90 degrees below horizontal. The optimal magnitude of the vector of translation of the humerus (30) similarly has a range, which is between 80% and 120% of the radius (28) of the humeral head (2) of the patient. In most cases the optimal magnitude of the vector of translation of the humerus (30) is about 100% of the radius (28) of the humeral head (2) of the patient.

Accordingly, disclosed is a prosthetic joint assembly for joining a humerus bone to a scapula bone, the humerus and scapula bones having a natural center of rotation relative to each other, the humerus bone having a humeral head diameter, the humerus bone being positionable with respect to the scapula bone between a rest position and an abducted position, the prosthetic joint assembly comprising a humeral component having two opposite ends, the first end comprising a humeral stem adapted for rigid engagement with the humerus bone and the second end comprising a concave dish; a scapular component having two opposite sides, the first side comprising a scapular base adapted for rigid engagement with the scapula bone and the second side comprising a convex surface adapted to engage the concave dish; wherein when the concave dish and the convex surface are engaged, the humeral component freely swivels with respect to the scapular component about a prosthetic center of rotation; wherein when the humeral stem is engaged with the humerus bone, the scapular stem is engaged with the scapula bone, and the concave dish and the convex surface are engaged, the prosthetic center of rotation is displaced in a direction that is inferior and medial relative to the natural center of rotation; wherein when the humeral stem is engaged with the humerus bone, the scapular stem is engaged with the scapula bone, and the concave dish and the convex surface are engaged, with the humerus in the rest position, the humerus bone is displaced in a direction that is inferior relative to the natural center of rotation; wherein the direction of displacement of the humerus bone is between 75 and 105 degrees below horizontal; wherein the ratio of the inferior displacement of the prosthetic center of rotation to the medial displacement of the prosthetic center of rotation is in the range between 0.6 and 1.2 (30-50 degrees below horizontal), preferably in the range of 0.85 to 1.15 (40-49 degrees below horizontal), and optimally equal to 1 (45 degrees below horizontal); wherein the distance of displacement of the prosthetic center of rotation relative to the natural center of rotation is between 60% and 80%, and optimally equal to 70%, of the radius of the humeral head; and wherein the distance of displacement of the humerus bone relative to the natural center of rotation is between 80% and 120%, and optimally equal to 100%, of the radius of the humeral head.

Also disclosed is a prosthetic joint assembly for joining a humerus bone to a scapula bone, the humerus and scapula bones having a natural center of rotation relative to each other, the humerus bone having a humeral head diameter, the humerus bone being positionable with respect to the scapula bone between a rest position and an abducted position, the prosthetic joint assembly comprising a humeral component having two opposite ends, the first end comprising a humeral stem adapted for rigid engagement with the humerus bone and the second end comprising a concave surface; a scapular baseplate having a vertical dimension and two opposite sides, the first side adapted for rigid engagement with the scapula bone, and the second side comprising a trunnion, the trunnion being offset inferiorly relative to a center of the vertical dimension; a glenosphere component having two opposite sides, the first side comprising an aperture adapted for rigid engagement with the trunnion, and the second side comprising a convex surface adapted to engage the concave surface; wherein when the concave surface and the convex surface are engaged the humeral component freely swivels with respect to the glenospheres about a prosthetic center of rotation. In this embodiment, the humeral component optionally comprises a stem component having a longitudinal axis and two opposite ends, the first end comprising the humeral stem, and the second end comprising a coupler interface; a coupler component having two opposite ends, the first end comprising a stem interface adapted to rigidly engage the stem component's coupler interface, and the second end comprising a cup interface; and a cup component having two opposite sides, the first side comprising a coupler interface adapted to rigidly engage the coupler component's cup interface, and the second side comprising the concave surface.

Also disclosed is a method for prosthetically joining a humerus bone to a scapula bone, the humerus and scapula bones having a natural center of rotation relative to each other, the humerus bone having a humeral head diameter, the humerus bone being positionable with respect to the scapula bone between a rest position and an abducted position, the method comprising the steps of (1) rigidly engaging a scapular component to the scapula bone; (2) rigidly engaging a humeral component to the humerus bone, the humeral component adapted to engage, and freely swivel with respect to, the scapular component about a prosthetic center of rotation; (3) wherein upon engagement of the humeral component to the scapular component, the prosthetic center of rotation is displaced in a direction that is inferior and medial relative to the natural center of rotation; (4) wherein upon engagement of the humeral component to the scapular component in the rest position, the humerus bone is displaced in a direction that is inferior relative to the natural center of rotation; (5) wherein the direction of displacement of the humerus bone is between 75 and 105 degrees below horizontal, and optimally 90 degrees below horizontal; (6) wherein the ratio of the inferior displacement of the prosthetic center of rotation to the medial displacement of the prosthetic center of rotation is in the range between 0.6 and 1.2 (30-50 degrees below horizontal), preferably in the range of 0.85 and 1.15 (40-49 degrees below horizontal); and optimally equal to 1 (45 degrees below horizontal); (7) wherein the distance of displacement of the prosthetic center of rotation relative to the natural center of rotation is between 60% and 80%, and optimally 70%, of the radius of the humeral head; and (8) wherein the distance of displacement of the humerus bone relative to the natural center of rotation is between 80% and 120%, and optimally equal to 100%, of the radius of the humeral head.

Further disclosed in another embodiment is a method for prosthetically joining a humerus bone to a scapula bone, the humerus bone comprising a medullar canal, a humeral head, and a humeral head radius distance, the humerus bone having a natural center of rotation (nCOR) with respect to the scapula bone. The nCOR representing that of a healthy joint. In at least one embodiment, the method comprises the steps of: providing a humeral component having two opposite ends, the first end optionally comprising a humeral stem adapted for rigid engagement with the medullar canal of the humerus bone and the second end optionally comprising a concave dish; providing a scapular component having two opposite sides, the first side optionally comprising a scapular baseplate adapted for rigid engagement with the scapula bone, the second side optionally comprising a convex surface adapted to engage the concave dish; relocating the humerus bone with respect to the scapula bone by (a) establishing a humeral reference point in a direction that is 30 to 60 degrees above horizontal and medial to the nCOR and a distance from the nCOR approximately equal to between 60% and 80% of the humeral head radius distance; (b) establishing a scapular reference point that is in a direction from the nCOR that is approximately normal (i.e., within 15 degrees of normal) and medial to the direction of the humeral reference point, and at a distance from the nCOR approximately equal to between 60% and 80% of the humeral head radius distance; (c) repositioning the humerus bone so that the humeral reference point is superimposed on the scapular reference point and establishing a prosthetic center of rotation (pCOR) at the scapular reference point; resecting the humeral head and engaging the humeral stem of the humeral component to the medullar canal of the repositioned humerus bone; engaging the scapular baseplate to the scapula bone; and engaging the concave dish of the humeral component with the convex surface of the scapular component so that the relocated humerus bone freely swivels with respect to the scapula bone about the pCOR.

Also disclosed is another embodiment of a prosthetic joint assembly for joining a humerus bone to a scapula bone, the humerus bone having a humeral head radius distance, the humerus bone having a natural center of rotation (nCOR) with respect to the scapula bone. The nCOR representing that of a healthy joint. In such embodiments, the prosthetic joint assembly may comprise a humeral component having two opposite ends, the first end comprising a humeral stem adapted for rigid engagement with the humerus bone and the second end comprising a concave dish; a scapular component having two opposite sides, the first side comprising a scapular baseplate adapted for rigid engagement with the scapula bone, the second side comprising a convex surface adapted to engage the concave dish; wherein the geometry of the humeral component and the scapular component is such that when humeral stem is engaged with the humerus bone, the scapular baseplate is engaged with the scapula bone, and the concave dish and the convex surface are engaged, the humerus bone freely swivels with respect to the scapular component about a prosthetic center of rotation (pCOR), and the humerus bone is shifted to a relocated position with respect to the scapula bone; and wherein the geometry of the humeral component and the scapular component is such that relocated position of the humerus bone with respect to the scapula bone is fixed by (a) calculating a humeral reference point in a direction that is 30 to 60 degrees above horizontal and medial to the nCOR and a distance from the nCOR approximately equal to between 60% and 80% the humeral head radius distance; (b) calculating a scapular reference point that is in a direction from the nCOR that is approximately normal (i.e., within 15 degrees of normal) and medial to the direction of the humeral reference point and at a distance from the nCOR approximately equal to between 60% and 80% the humeral head radius distance; (c) moving the humerus bone so that the humeral reference point is superimposed on the scapular reference point and establishing the pCOR at the scapular reference point.

Also disclosed is a baseplate for engagement with a scapula bone, the scapula bone having a glenoid cavity, the baseplate comprising: a vertical dimension; a first side optionally comprising a mating surface with a scapular stem, the mating surface optionally adapted to cooperate with the glenoid cavity, the scapular stem adapted for insertion into, and rigid engagement with, the scapula bone; and a second side opposite of the first side optionally comprising a trunnion offset inferiorly relative to a center of the vertical dimension, the trunnion being adapted to engage with a glenosphere component.

Although the invention is illustrated and described herein as embodied in a shoulder prosthesis, it is nevertheless not intended to be limited to only the details shown, since various modifications and structural changes may be made therein without departing from the spirit of the invention and within the scope and range of equivalents of the claims. Moreover, many of the principles and techniques discussed in the following description can be applied to prostheses used in other joints in the human anatomy.

The construction of the invention, together with additional objects and advantages thereof will be best understood from the following description of the specific disclosed embodiments when read in connection with the accompanying drawings.

### BRIEF DESCRIPTION OF THE DRA WINGS

FIG. 1 is an illustration of the bones of the human shoulder in the rest position provided for reference and to illustrate the principles of operation of the present invention.

FIG. 2 is an illustration of the bones of the human shoulder in the abducted position provided for reference and to illustrate the principles of operation of the present invention.

FIG. 3 is a close-up view of the interface between the human scapula and humerus bones illustrating the relocation of the center of rotation and humeral bone position in accordance with the present invention.

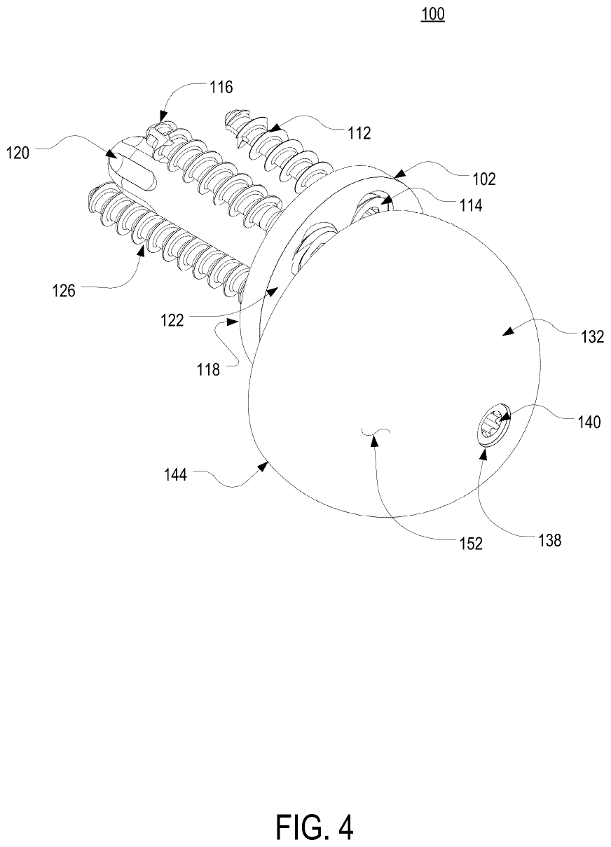

FIG. 4 is an isometric view of the scapular component of a shoulder prosthesis according to the present invention.

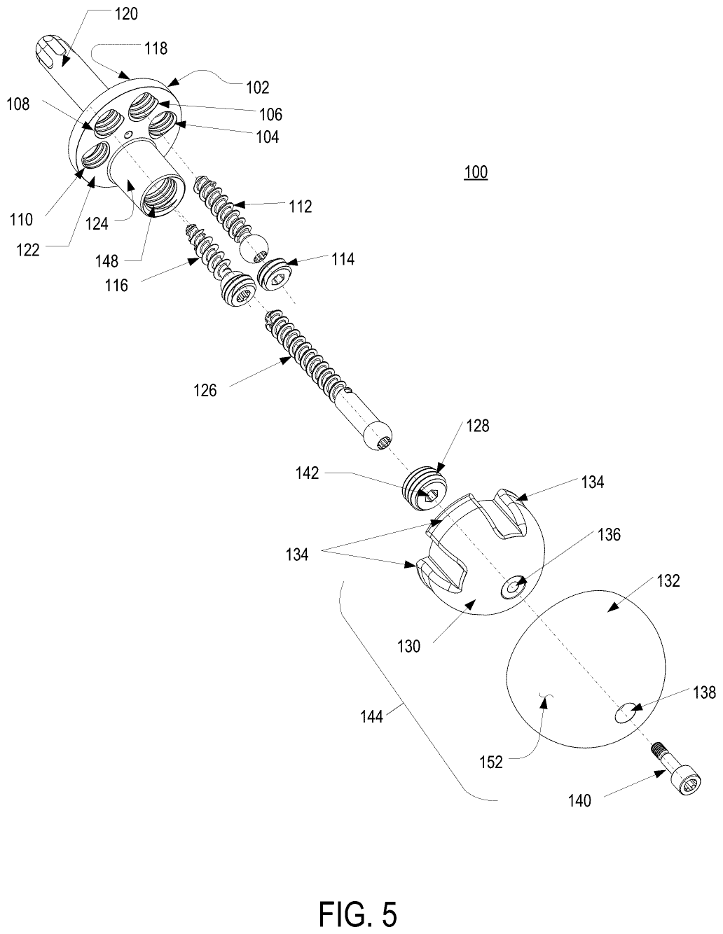

FIG. 5 is an exploded orthographic view of the scapular component of the shoulder prosthesis shown in FIG. 4.

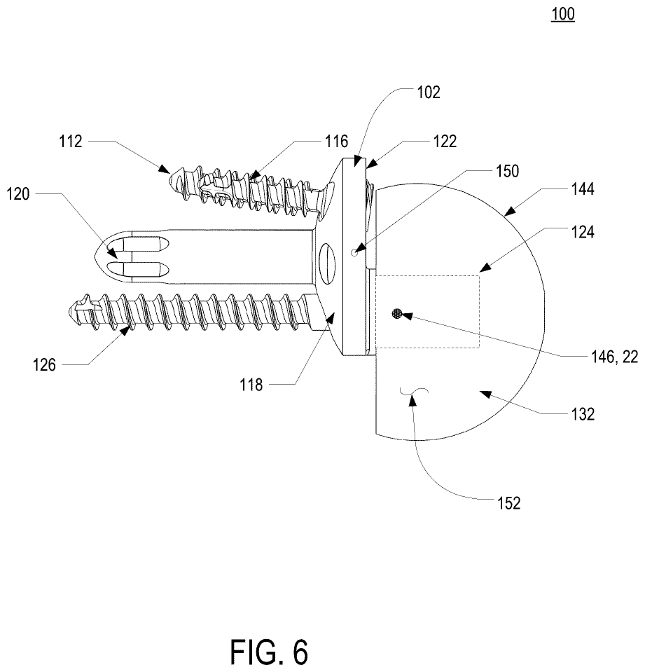

FIG. 6 shows a side view of the scapular component of a shoulder prosthesis shown in FIG. 4.

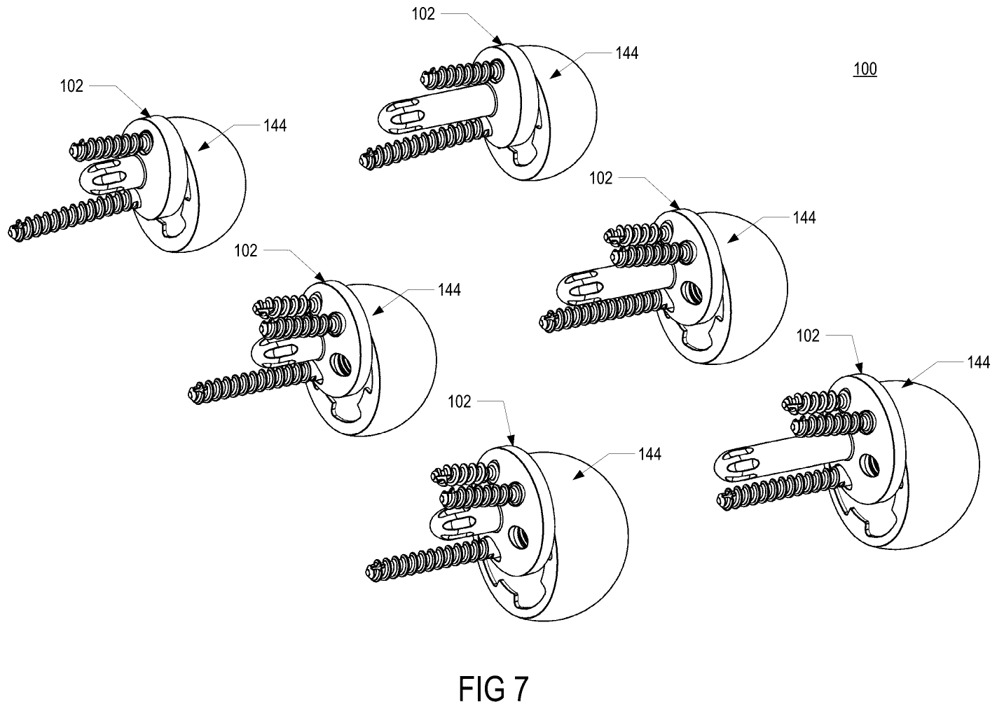

FIG. 7 shows multiple sized variations of the scapular component of a shoulder prosthesis according to the present invention.

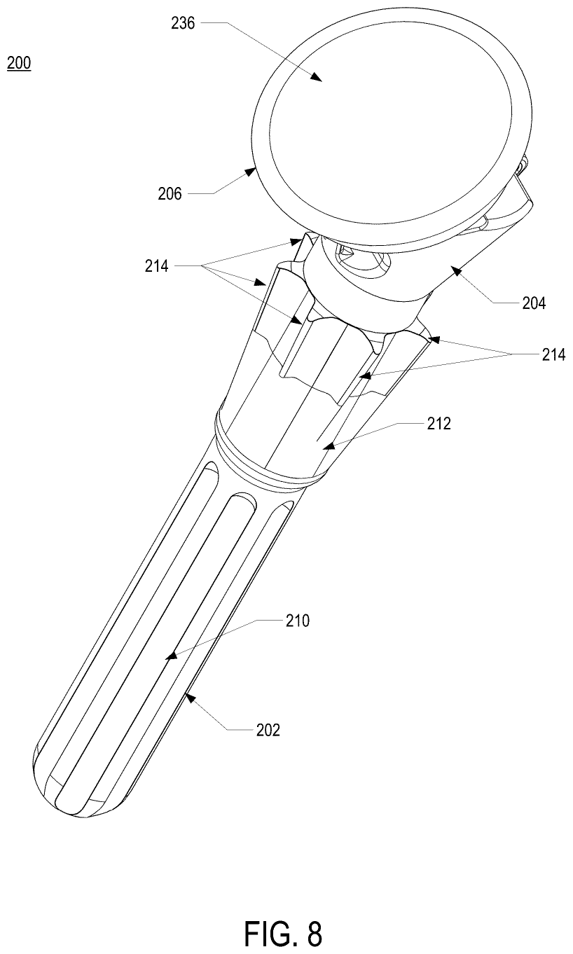

FIG. 8 is an isometric view of the humeral component of a shoulder prosthesis according to the present invention.

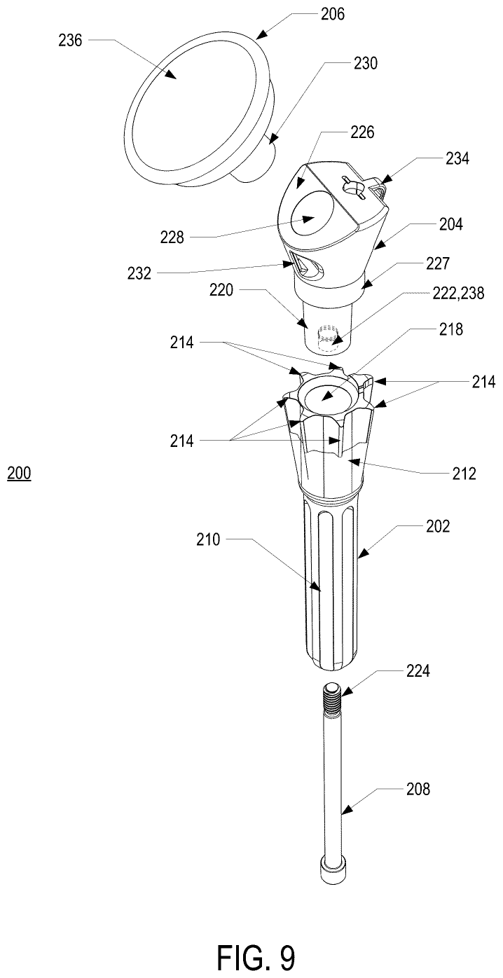

FIG. 9 is an exploded orthographic view of the humeral component of the shoulder prosthesis shown in FIG. 8.

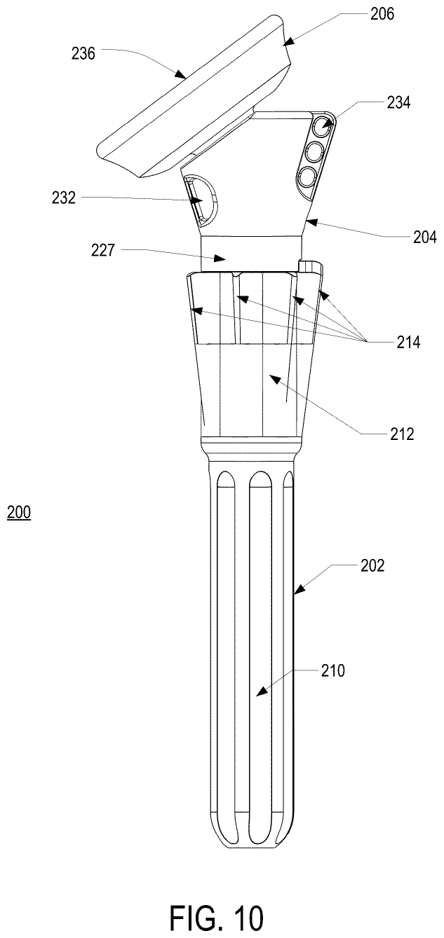

FIG. 10 shows a side view of the humeral component of a shoulder prosthesis shown in FIG. 8.

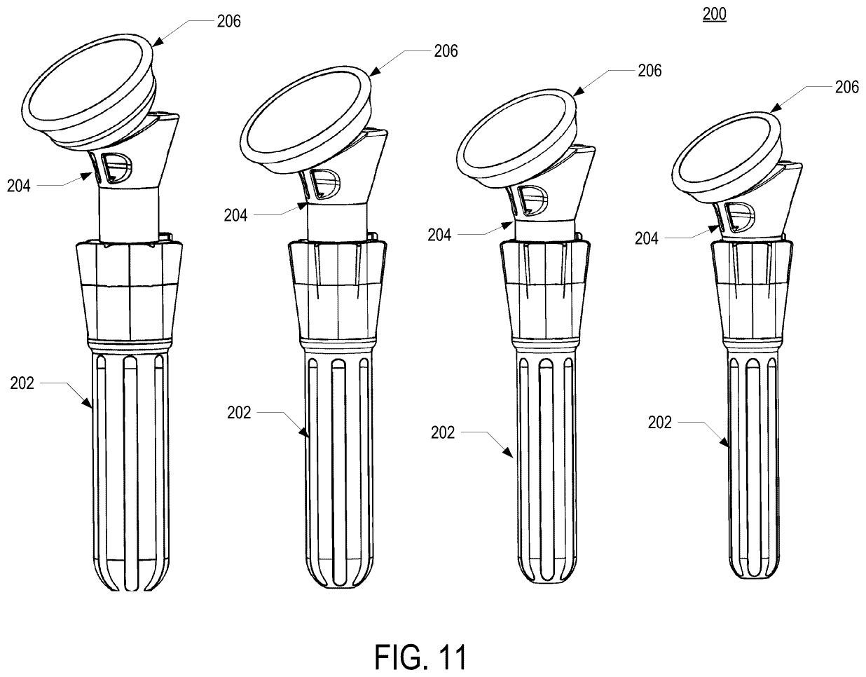

FIG. 11 shows multiple sized variations of the humeral component of a shoulder prosthesis according to the present invention.

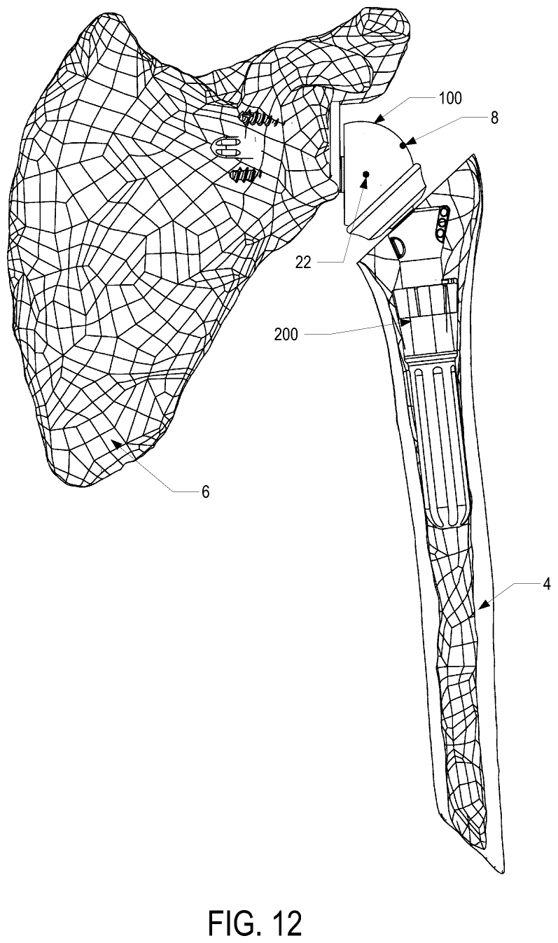

FIG. 12 shows, in the rest position, a shoulder prosthesis according to the present invention implanted on human scapula and humerus bones.

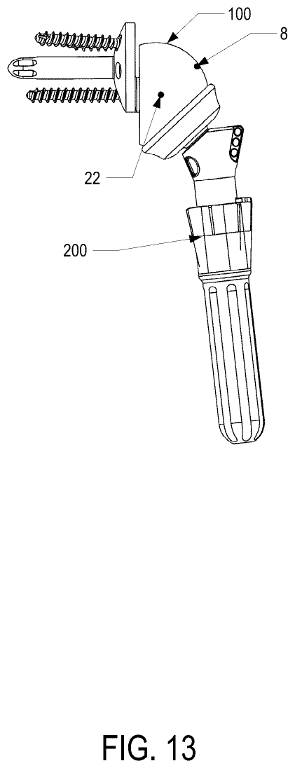

FIG. 13 shows the shoulder prosthesis of FIG. 12 with the humerus and scapula bones removed for clarity.

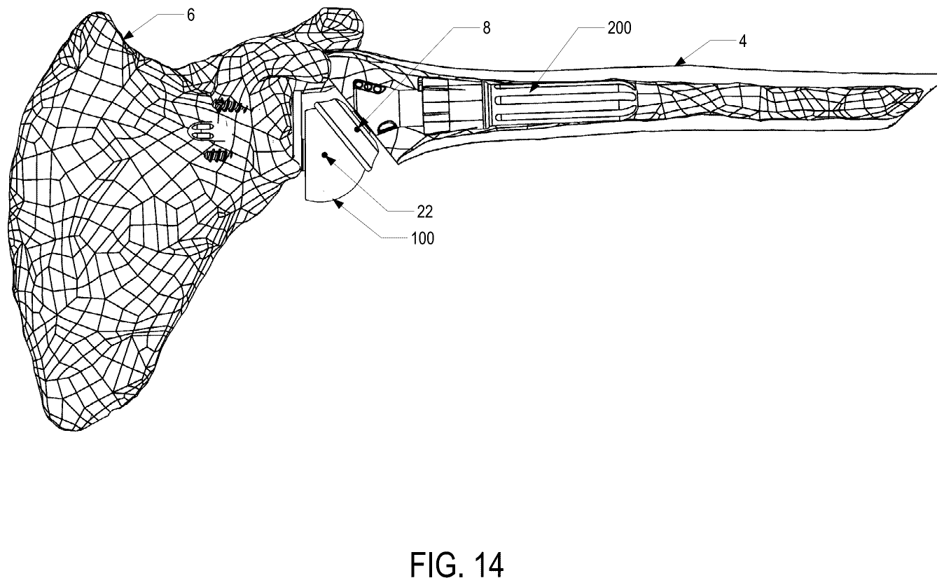

FIG. 14 shows, in the abducted position, a shoulder prosthesis according to the present invention implanted on human scapula and humerus bones.

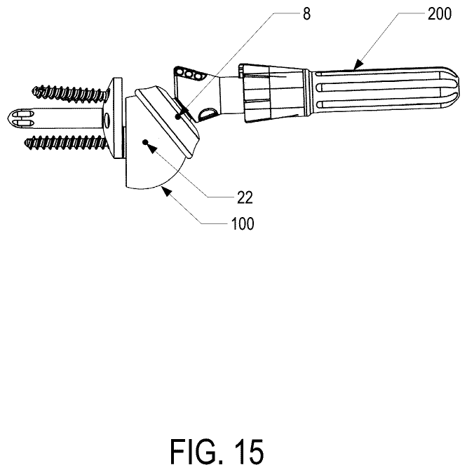

FIG. 15 shows the shoulder prosthesis of FIG. 14 with the humerus and scapula bones removed for clarity.

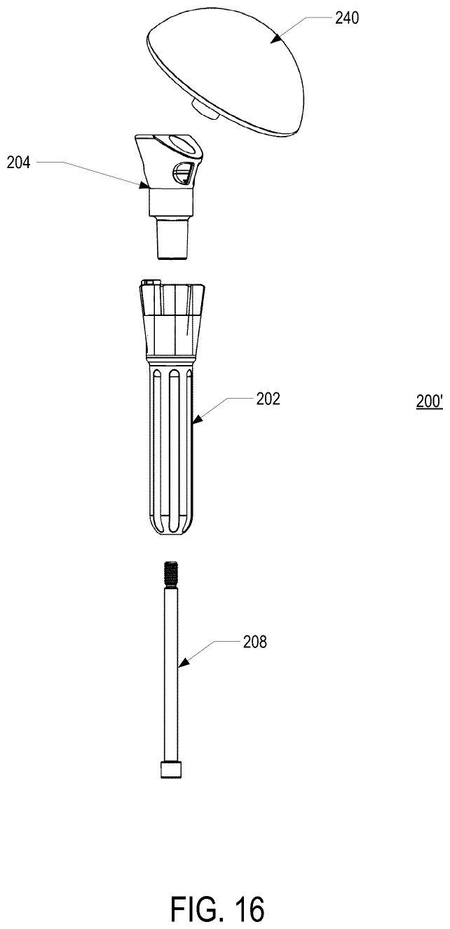

FIG. 16 shows an optional variation of a humeral component according to the present invention which is equipped with a large convex bearing element.

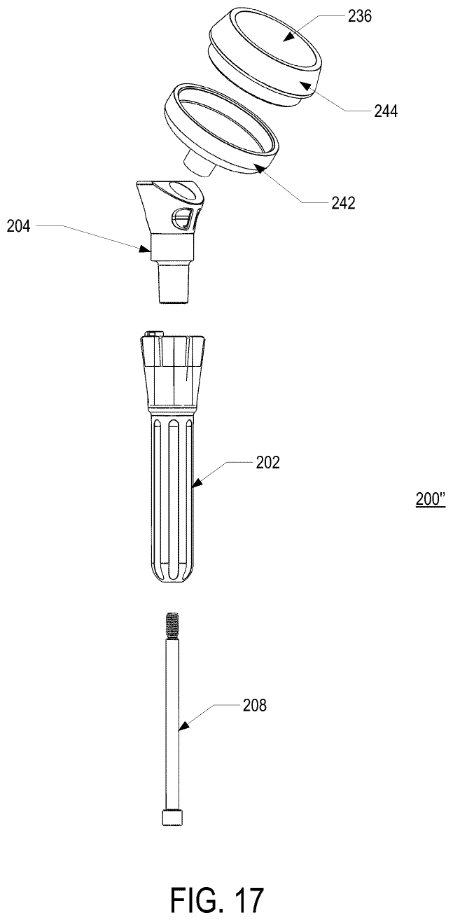

FIG. 17 shows an optional variation of a humeral component according to the present invention which is equipped with a two-piece concave bearing element.

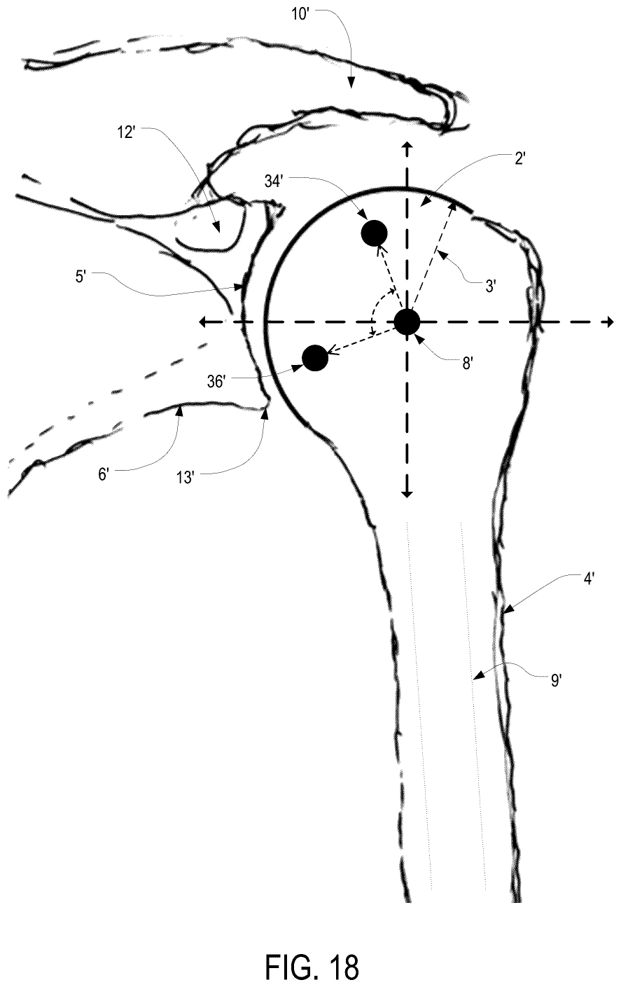

FIG. 18 is an illustration of a human humerus bone and scapula bone showing a scapular reference point and a humeral reference point for the relocation of a center of rotation of the humerus bone in accordance with another embodiment.

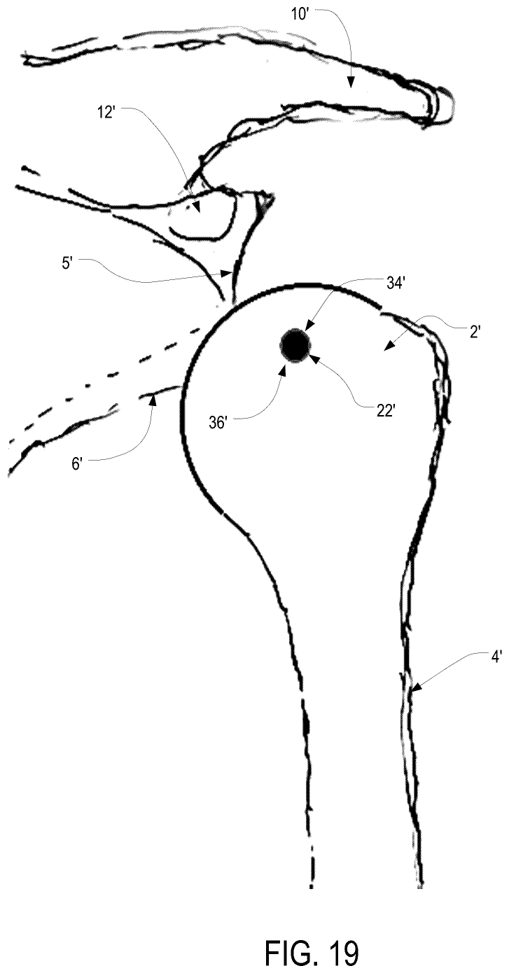

FIG. 19 is an illustration of a relocated human humerus bone with respect to a human scapula bone in accordance with one embodiment.

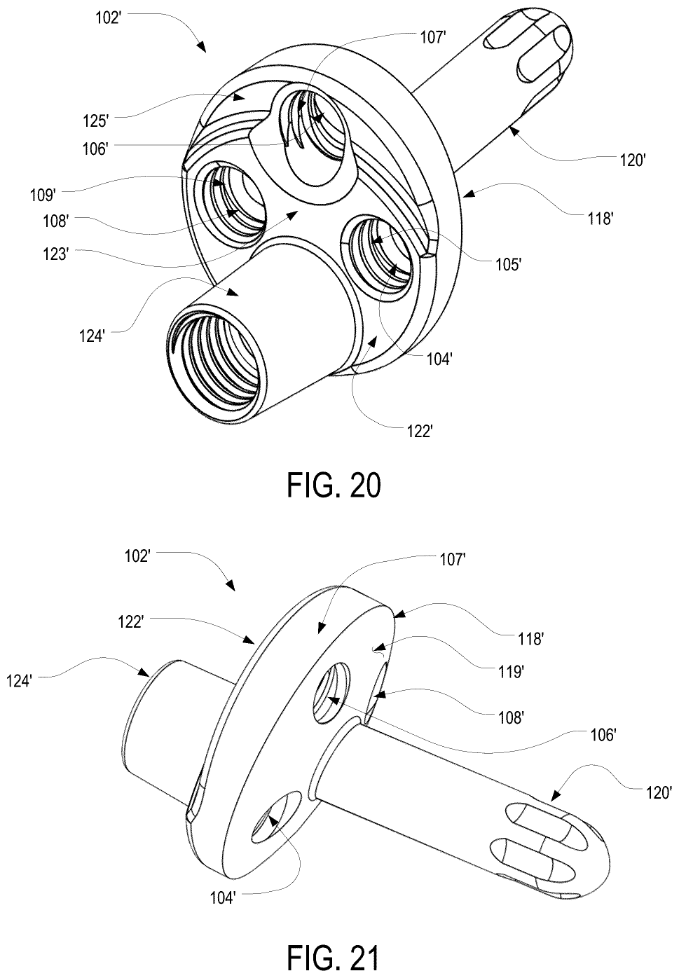

FIGS. 20 and 21 show front and back perspective views, respectively, of an optional variation of another scapular baseplate in accordance with another embodiment.

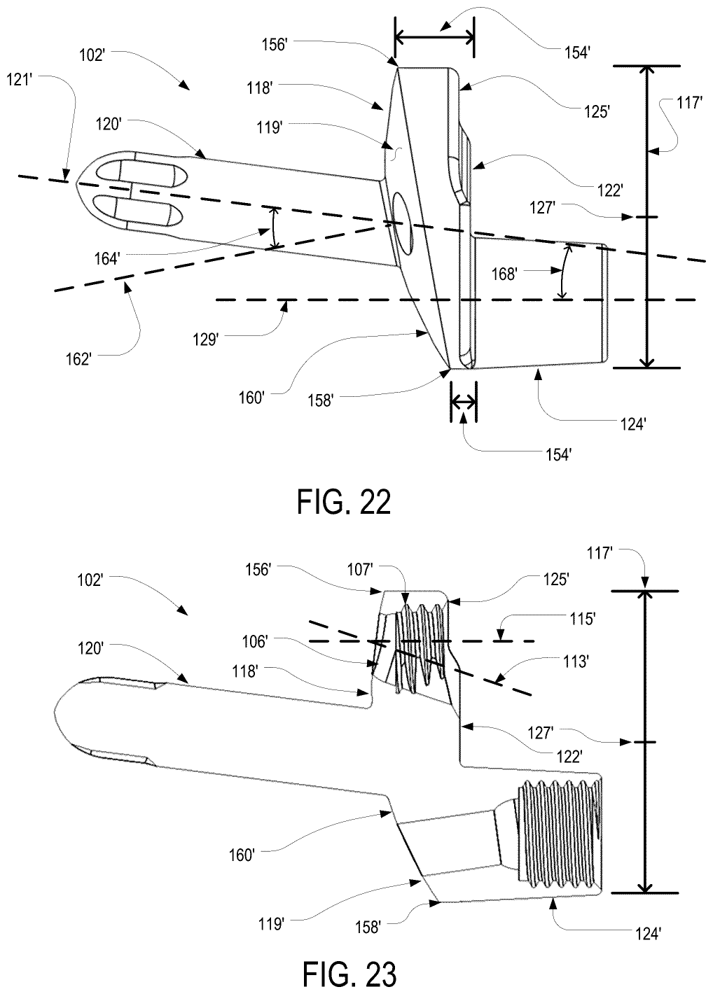

FIGS. 22 and 23 show side and cross-sectional side views, respectively, of the scapular baseplate of FIGS. 20 and 21, showing additional details in accordance with one embodiment.

FIG. 24 shows a top view of the scapular baseplate of FIGS. 20 through 23 showing additional details in accordance with one embodiment.

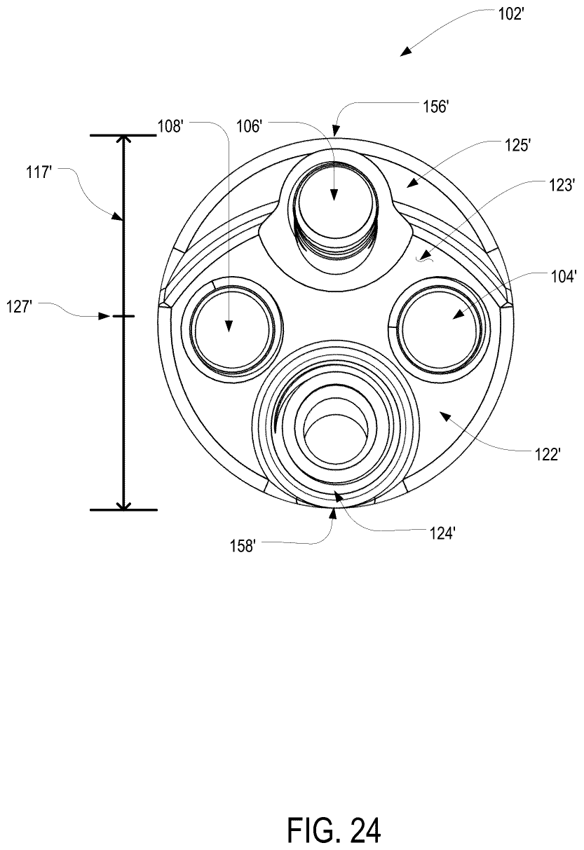

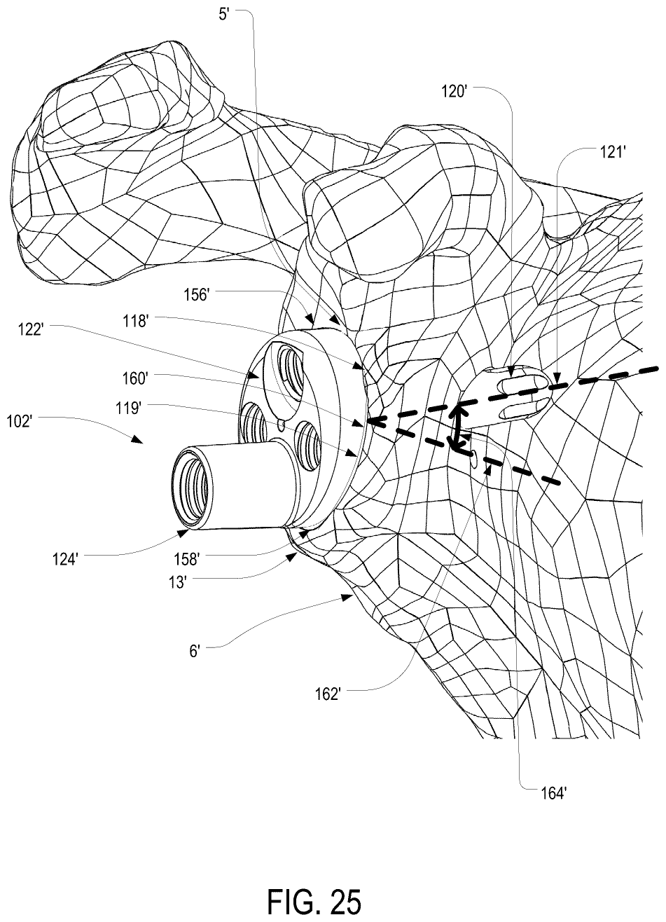

FIG. 25 illustrates a scapular baseplate having an optionally curved mating surface engaged with a human scapula bone in accordance with another embodiment.

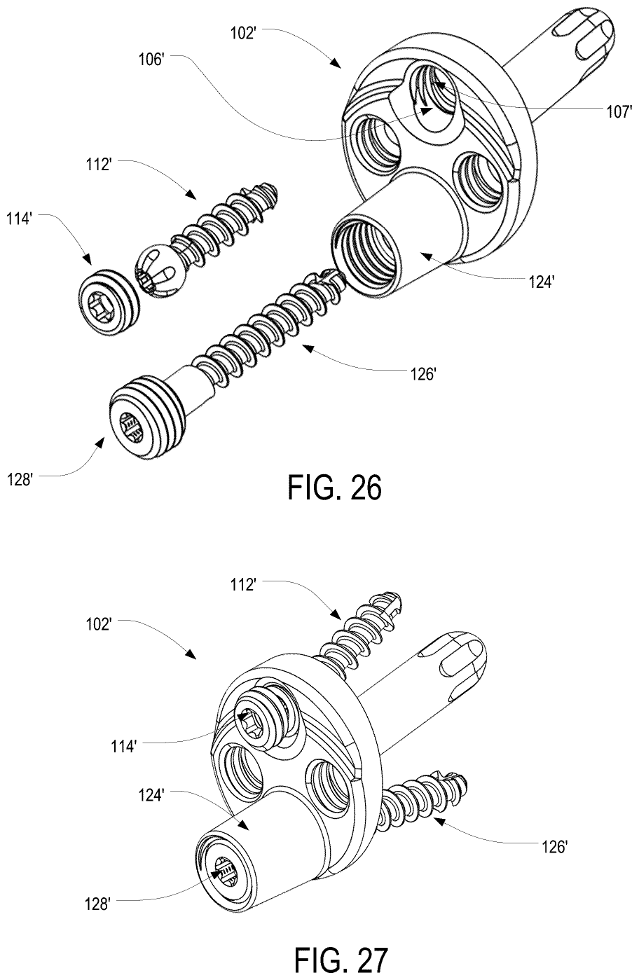

FIG. 26 shows an exploded view of a scapular baseplate and an optional baseplate polyaxial screw with a locking cap and an optional trunnion polyaxial screw with a trunnion locking cap in accordance with another embodiment.

FIG. 27 shows the scapular baseplate of FIG. 26 having received the optional baseplate polyaxial screw with locking cap at a hole of the baseplate and having received the optional trunnion polyaxial screw with trunnion locking cap at the trunnion in accordance with another embodiment.

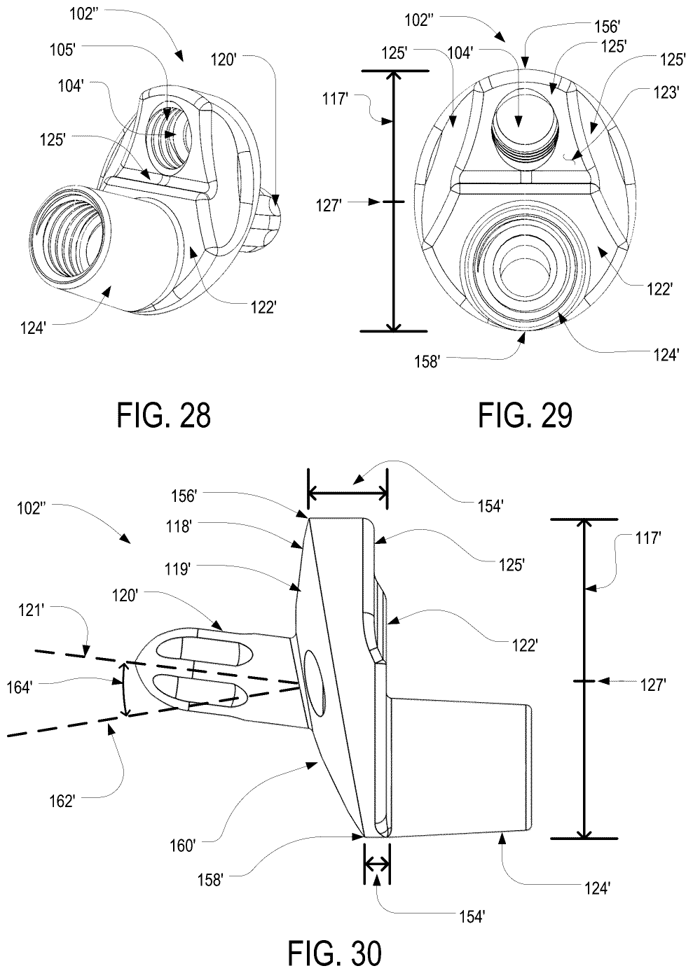

FIGS. 28, 29 and 30 show perspective, top and side views, respectively, of another optional variation of a scapular baseplate in accordance with another embodiment.

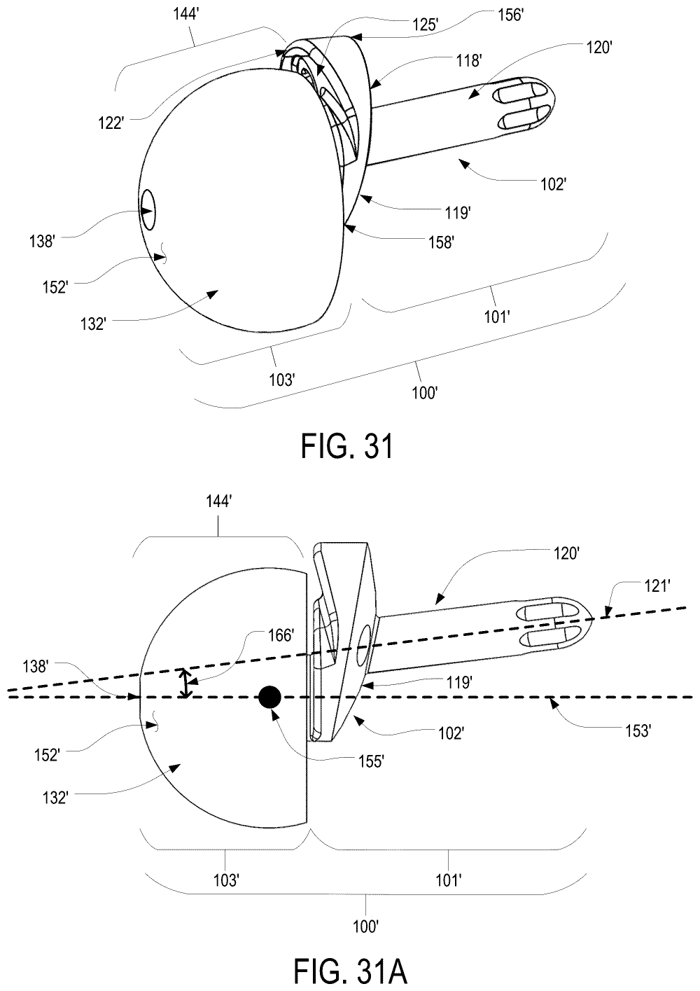

FIGS. 31 and 31A show perspective and side views, respectively of an optional variation of another scapular component, the scapular component having a scapular baseplate and a glenosphere component in accordance with another embodiment.

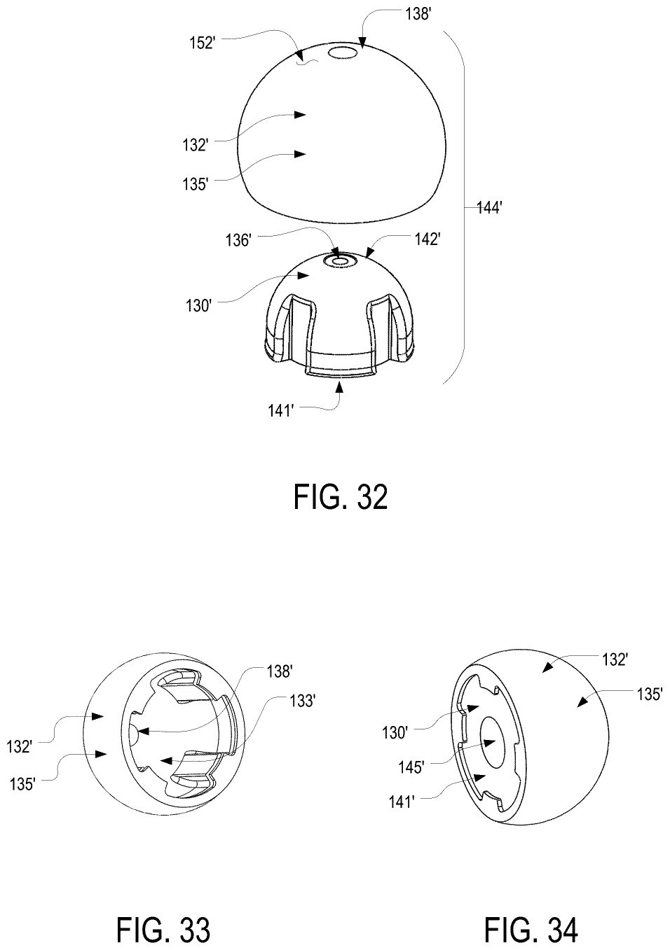

FIG. 32 shows an exploded view of a glenosphere component having a glenosphere core and a glensophere cover in accordance with another embodiment.

FIG. 33 shows a perspective view of a glenosphere cover in accordance with one embodiment.

FIG. 34 shows a perspective view of an assembled glenosphere component in accordance with another embodiment.

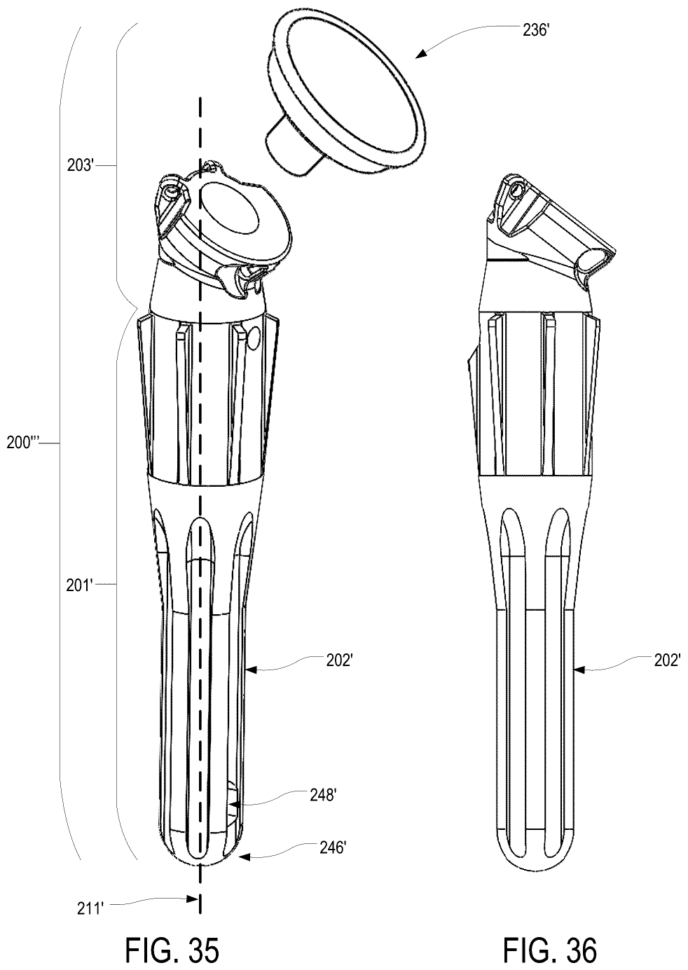

FIG. 35 shows an exploded perspective view of an optional variation of another humeral component, the humeral component having a humeral stem and a concave dish in accordance with another embodiment.

FIG. 36 shows a side view of the humeral stem in accordance with one embodiment.

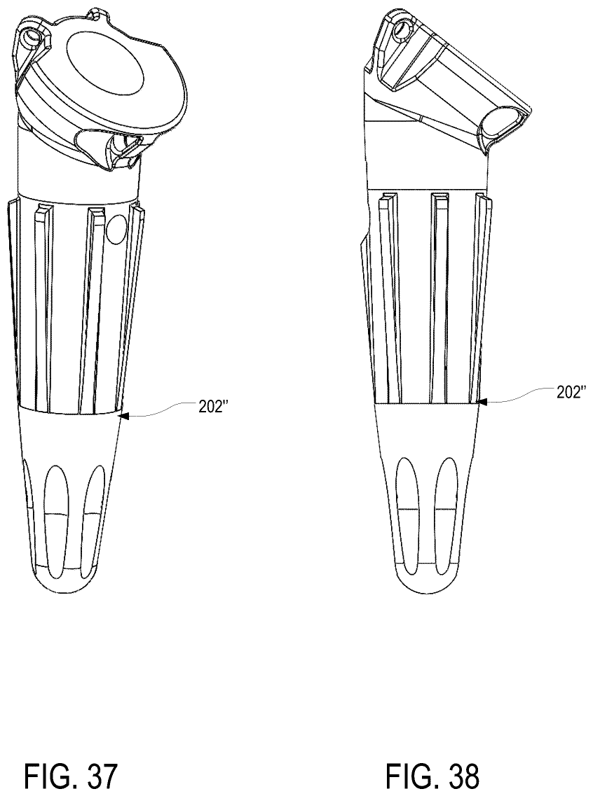

FIGS. 37 and 38 show perspective and side views, respectively, of another optional variation of a humeral stem in accordance with another embodiment.

### DETAILED DESCRIPTION OF THE INVENTION

An object of the prosthesis herein disclosed is to achieve the optimal final placement of the pCOR and the humerus bone as explained in detail in the previous sections and as illustrated in FIG. 3. FIG. 3 is a close-up illustration of the interface between the humerus and scapula showing the vectors of translation, and final placement, of the pCOR and the humerus bone. This optimal positioning is achieved by utilizing a scapular component (100) and humeral components (200) described in detail in the following paragraphs.

Referring to FIGS. 4, 5, and 6 shown are, respectively, isometric, exploded, and side views fully describing the construction of the scapular component (100). Scapular component (100) comprises a base (102) which is adapted for rigid engagement to the glenoid (5) of the scapular bone (6) through one or more screw holes (104, 106, 108, 110). The screw holes are adapted to receive one or more polyaxial locking screws (112) with corresponding locking caps (114) or single-axis locking screws (116) to affix base (102) to the glenoid (5). The back side (118) of base (102) may optionally include a stem (120) adapted to be implanted into the glenoid (5) to provide additional torsional support for the scapular component (100).

The front side (122) of base (102) includes trunnion (124) of approximately cylindrical construction. The trunnion (124) can optionally be bored and include an internal thread (148) which is adapted to receive a single-axis locking screw (not shown) or a polyaxial locking screw (126) and a corresponding locking cap (128). The external surface of the trunnion (124) is adapted to receive a glenosphere core (130) which, in turn, is adapted to receive a hollow glenosphere cover (132). The glenosphere core (130) has one or more lobes (134) that closely correspond to matching apertures in the glenosphere cover (132), which ensure that the glenosphere cover (132) does not rotate with respect to the glenosphere core (130) once assembled. When assembled, the glenosphere core (130) and glenosphere cover (132) comprise a spherically shaped glenosphere assembly (144) with an outside surface (152) adapted to interface with humeral component (200).

The glenosphere core (130) and glenosphere cover (132) feature screw holes (136, 138) that are aligned with trunnion (124) and locking cap (128), and are adapted to receive setting screw (140) to secure the glenosphere assembly (144) to the base (102). The back of locking cap (128) is equipped with internal threads (142) that correspond with the external threads of setting screw (140) for that purpose. In the event that the trunnion (124) is not bored and therefore does not accept polyaxial locking screw (126) and locking cap (128), a hole with internal threads can be placed at the tip of the trunnion (124) to engage with setting screw (140). Although a two-piece glenosphere assembly (144) is shown in the described embodiment, it should be understood that a single piece glenosphere (not shown) can optionally be used with similar effectiveness.

With the exception of the glenosphere cover (132), all of the components of the scapular component (100) are, preferably, of metallic composition, such as, without limitation, biocompatible surgical-grade alloys like cobalt-chromium-molybdenum (“CoCrMo”) or Titanium alloys well suited for biomedical applications such as joint replacements. Glenosphere cover (132) is manufactured from a durable yet resilient plastic material such as, without limitation, ultra-high-molecular-weight polyethylene (“UHMWPE.”) If a single piece glenosphere is used, it can be of metallic or plastic construction.

As will be readily observed, trunnion (124) is located well inferior to the center (150) of base (102). This ensures that the center (146) of the glenosphere assembly (144) which will become the prosthetic center of rotation (22), is well inferiorized. Moreover, because center (146) of the glenosphere assembly (144) is located very close to base (102) it is also well medialized. As discussed previously, inferiorization and medialization of the prosthetic center of rotation (22) with respect to the natural center of rotation (8) is one of the primary objectives achieved by the described arrangement of components.

Referring next to FIG. 7, shown are multiple sized variations of the scapular component (100) which can be utilized depending on the anatomy of the patient and the magnitude and angle of translation of the pCOR (22) desired by the surgeon. As will be seen, some of the sizes include a single attachment screw and corresponding base hole, while others include up to 4 holes and screws. In addition, the shape of the base (102) varies from circular to oval shaped. It should be observed that additional base (102) shapes can be used without departing from the principles of the disclosed invention. The described modular arrangement permits the use of differently sized glenosphere assemblies (144) with differently sized and shaped bases (102) to assemble a scapular component (100) which is optimally adapted to the anatomy of the patient and the desired prosthetic center of rotation (22) location.

Referring to FIGS. 8, 9, and 10, shown are, respectively, isometric, exploded, and side views fully describing the construction of the humeral component (200). Humeral component (200) comprises a stem (202), a coupler (204), cup (206), and assembly screw (208). Stem (202) is a substantially elongated member comprising a medular stem (210) at one end, and a cone (212) at the other end. Stem (202) is bored through its longitudinal axis to permit assembly screw (208) to enter through the medular stem (210) end and engage coupler (204) at the cone (212) end. Medular stem (210) comprises a typical bone stem adapted to penetrate the medullary canal of the humerus bone (4) and rigidly engage the bone. Medular stem (210) can be adapted for cement or cementless applications. Cone (212) gradually expands in diameter and terminates in one or more fins (214) that are adapted to engage trabecular bone in the humerus bone (4) to transmit torsional loads and prevent stem (202) from rotating once implanted. The top portion of cone (212) comprises a stem shaft opening (218) adapted to receive coupler (204)

Coupler (204) comprises a stem engagement shaft (220) adapted to engage stem shaft opening (218) and form a secure interference or press fit between stem (202) and coupler (204). In addition, the bottom surface of stem engagement shaft (220) comprises an opening (222) with internal threads (238) that receive the threads (224) of assembly screw (208) after it is inserted through the bottom of the medular stem (210) end of stem (202). In one embodiment stem engagement shaft (220) and shaft opening (218) form a morse taper which provides for a secure frictional fit. In order to make the engagement between the stem (202) and coupler (204) even more secure against torsional forces, stem engagement shaft (220) can optionally be offset from the centerline of stem (202). The combination of a morse taper and the off-center location of stem engagement shaft (220) provide for an extremely robust and torsion resistant fit between stem (202) and coupler (204) once assembly screw (208) is tightened.

The top surface (226) of coupler (204) comprises a slanted landing area with an opening to receive cup (206). The angle of slant of top surface (226) provides the appropriate angle for displacement of the humerus (4) in the lateral-inferior direction in relation to the pCOR (22) once the prosthesis is assembled. An intermediate coupler section (227) provides additional inferiorization of the humerus bone (4) with respect to the pCOR (22) should it be necessary to achieve optimal placement of the humerus. A cup shaft opening (228) on top surface (226) is adapted to receive cup shaft (230) to secure cup (206) to coupler (204). Cup shaft opening (228) and cup shaft (230) may comprise another morse taper to ensure secure engagement between coupler (204) and cup (206). Additionally, coupler (204) may include one or more medial (232) and lateral (234) suture attachment points.

One end of cup (206) comprises a concave surface, or dish (236) which closely matches, and is adapted to engage, the outside surface (152) of glenosphere assembly (144) (see FIGS. 4-6.) The other end of cup (206) comprises cup shaft (230) which, as previously discussed, engages in an interference fit, and optionally a morse taper or other type of shallow angle self-holding taper, with cup shaft opening (228) to securely engage cup (206) to coupler (204).

All of the components of the humeral component (200) are, preferably, of metallic composition, such as, without limitation, biocompatible surgical-grade alloys like cobalt-chromium-molybdenum (“CoCrMo”) or Titanium alloys well suited for biomedical applications such as joint replacements.

Referring next to FIG. 11, shown are multiple sized variations of the humeral component (200) which can be utilized depending on the anatomy of the patient and the magnitude and angle of further translation of the humerus bone (4) desired by the surgeon. As will be seen, the various sizes include stems (202) of different diameters, cones (212) with varying tapers, intermediate coupler sections (227) of varying lengths, and concave surfaces or dishes (236) of different diameters and curvatures to match corresponding glenosphere assemblies (144). The described modular arrangement permits the use of differently sized stems (202) with differently sized couplers (204) and cups (206) to assemble a humeral component (200) which is optimally adapted to the anatomy of the patient and the optimal displacement of the humerus bone (4) with respect to the prosthetic center of rotation (22).

The procedure for implanting the disclosed prosthetic shoulder on a patient includes the following generalized steps. First, the size and relative position of the humerus (4), humeral head (2), scapula (6), glenoid (5), and natural center of rotation (8) of the patient's anatomy are measured. Next, based on these measurements, a scapular component (100), and a humeral component (200), are assembled using the various modular elements, including appropriately sized base (102), glenosphere assembly (144), stem (202), coupler (204), and cup (206) elements. Then, the glenoid (5) is prepared to receive the scapular component (100) which is implanted at the appropriate location to achieve the desired level of inferiorization of the pCOR (22). Next, the humeral head is removed from the humerus (4) and the humeral component (200) is implanted in its place. Finally the scapular component (100) and humeral component (200) are mated and the shoulder joint is tested going from the at rest position to the abducted position and back. If any impingement is detected between the humerus and scapula one or more of the modular elements of the scapular component (100) or the humeral component (200) can be replaced to achieve an optimal alignment of the shoulder joint.

FIGS. 12 and 13 show views of the presently disclosed prosthetic shoulder joint, fully assembled and implanted in the “at rest” position. Shown in FIG. 12 is an assembled shoulder prosthesis according to the present invention implanted on human scapula (6) and humerus (4) bones. FIG. 13 shows the same assembled prosthesis, including the scapular component (100) and the humeral component (200), with the bones not shown for added clarity. It should be noted that the prosthetic center of rotation (22) is significantly shifted in the medial and inferior direction with respect to the natural center of rotation (8) and that the humerus bone (4) is similarly significantly further shifted in the inferior direction with respect to the natural center of rotation (8). This arrangement positions the humerus bone (4) in the optimal placement for full rotation to the abducted position with minimized risk of impingement on any features of the scapula (6).

Next FIGS. 14 and 15 show views of the presently disclosed prosthetic shoulder fully assembled and implanted after rotation to the “abducted” position. Shown in FIG. 14 is an assembled shoulder prosthesis according to the present invention implanted on human scapula (6) and humerus (4) bones. FIG. 15 shows the same assembled prosthesis, including the scapular component (100) and the humeral component (200), with the bones not shown for added clarity. As will be appreciated, the prosthetic center of rotation (22) remains significantly shifted in the medial and inferior direction with respect to the natural center of rotation (8). In the abducted position, the humerus bone (4) is now in almost the same position as it would be in a healthy shoulder (see FIG. 2) reflecting the almost complete restoration of the range of movement of the shoulder.

Variations of the disclosed prosthetic joint are also possible as needed for special situations that may arise from time to time. One such situation occurs when after performing a total reverse shoulder arthroplasty using the disclosed prosthesis it is determined that the patient is no longer a suitable candidate to continue using the reverse shoulder prosthesis. This situation could arise due to, for example, failure of the scapula to support the scapular element (100), as a result of re-injury, or due to degenerative changes in the patient. In such a situation, the scapular element can be removed, and the humeral element can be modified to provide a glenosphere, instead of a cup, to interface with the natural glenoid. This avoids having to completely replace the humeral component, a procedure that could be difficult and/or traumatic to the patient. FIG. 16 shows an optional variation of a humeral component (200′) to address such a situation. As shown in this figure, the cup element (206) of the humeral component has been removed, and in its place a large glenosphere (240) is attached to the coupler (204).

FIG. 17 shows an alternative embodiment of the humeral component (200″) in which the concave bearing surface is manufactured from plastic material, while the remaining parts are metallic. This is achieved by replacing cup (206) with a two-piece component consisting of a metallic tray (242) and a cooperating plastic cup insert (244) which comprises a concave surface.

Shown throughout FIGS. 18 through 38 are various illustrations in accordance with other embodiments of a prosthetic joint assembly for joining a humerus bone (4′) to a scapula bone (6′). It should be understood that these embodiments are not restrictive and that the features of these embodiments may be modified, combined, and/or substituted with features of the previously described embodiments as would be apparent to those of ordinary skill in the art. Referring to FIG. 18, shown is an illustration of a human humerus bone (4′) and scapula bone (6′). As described previously, the humerus bone (4′) has a medullar canal (9′), a humeral head (2′), and a humeral head radius distance (3′). Shown in the illustration of FIG. 18, the scapula bone (6′) has a glenoid cavity (5′), acromion (10′) and coracoid process (12′). The glenoid cavity (5′) has an inferior edge (13′). The humerus bone (4′) further comprises a natural center of rotation (nCOR) (8′) with respect to the scapula bone (6′). The nCOR (8′) represents that of a healthy joint. In at least one embodiment, the prosthetic joint assembly may comprise a humeral component (200′″), such as the one depicted in FIG. 35, and a scapular component (100′), such as the one depicted in FIG. 31.

Referring again to FIG. 35, the humeral component (200′″) may comprise two opposite ends, the first end (201′) optionally comprising a humeral stem (202′) adapted for rigid engagement with the humerus bone (4′) and the second end (203′) optionally comprising a concave dish (236′). Referring to FIGS. 35 through 38, it is to be understood that the humeral stem may be of a variety of lengths, shapes and configurations. For example, referring to FIGS. 37 and 38, the humeral stem (202″) may be a shorter length. A shorter humeral stem (202″) may be desirable in certain cases, such as for arthritic applications. Referring again to FIGS. 35 and 36, in at least some embodiments the humeral stem (202′) may comprise a single, unitary component. Referring further to FIG. 35, in some embodiments a bottom end (246′) of the humeral stem (202′) may optionally comprise a hole (248′) that extends through the humeral stem (202′) transverse to a longitudinal axis (211′) of the humeral stem (202′). The hole (248′) may be adapted to receive a screw to rigidly engage the humeral component (200″) to the humerus bone (4′) (engagement not shown).

Referring to FIGS. 31 and 31A, the scapular component (100′) may comprise two opposite sides, the first side (101′) optionally comprising a scapular baseplate (102′) having a scapular stem (120′) and the second side (103′) may optionally comprise a convex surface (152′) having a central axis (153′). In the present context, the “central axis” (153′) of the convex surface (152′) refers to an axis that extends from the geometric center (155′) of convex surface (152′) and a point on the center of the convex surface (i.e. glenosphere hole (138′) referenced below). In some embodiments, the scapular stem (120′) comprises a longitudinal axis (121′) that extends outward at an angle (166′) between 3 and 15 degrees superior to the central axis (153′) of the convex surface (152′). In some embodiments, the longitudinal axis (121′) of the scapular stem (120′) is at an angle (166′) equal to 7 degrees superior to the central axis (153′) of the convex surface (152′). The convex surface (152′) may be adapted to engage the concave dish (236′) (see, for example, FIG. 35) of the humeral component (200′″). Referring to FIG. 25, the scapular baseplate (102′) may be adapted for rigid engagement with the scapula bone (6′).

In accordance with an embodiment of the prosthetic joint assembly, the geometry of the humeral component (200′″) and the scapular component (100′) is such that when humeral stem (202′) is engaged with the humerus bone (4′), the scapular baseplate (102′) is engaged with the scapula bone (6′), and the concave dish (236′) and the convex surface (152′) are engaged, the humerus bone (4′) freely swivels with respect to the scapular component (100′) about a prosthetic center of rotation (pCOR) (22′) (see pCOR (22′) in FIG. 19), and the humerus bone (4′) is shifted to a relocated position with respect to the scapula bone (6′). FIG. 18 illustrates a humeral bone (4′) and scapula bone (6′) before relocation. FIG. 19 illustrates the relocated position of the humerus bone (4′) with respect to the scapula bone (6′), without the prosthetic joint assembly engaged, in accordance with one embodiment. The prosthetic joint assembly when engaged with the scapula bone (6′) and humerus bone (4′) may be adapted to shift the humerus bone to the relocated position, such as that depicted in FIG. 19. Referring to FIG. 18, in further accordance with an embodiment of the prosthetic joint assembly, the geometry of the humeral component (200′″) and the scapular component (100′) is such that relocated position of the humerus bone (4′) with respect to the scapula bone (6′) (as illustrated in FIG. 19) is fixed by (a) calculating a humeral reference point (34′) in a direction that is optionally 30 to 60 degrees above horizontal and medial to the nCOR (8′) and a distance from the nCOR (8′) approximately equal to between 60% and 80%, and optionally 70%, the humeral head radius distance (3′); (b) calculating a scapular reference point (36′) that is in a direction from the nCOR that is approximately normal (i.e., within 15 degrees of normal) and medial to the direction of the humeral reference point (34′) and at a distance from the nCOR (8′) approximately equal to between 60% and 80%, and optionally 70%, the humeral head radius distance (3′); (c) moving the humerus bone (4′) so that the humeral reference point (34′) is superimposed on the scapular reference point (36′) and establishing the pCOR (22′) at the scapular reference point (36′), as illustrated FIG. 19. In further embodiments, the distance of the humeral reference point (34′) from the nCOR (8′) is approximately equal to the distance of the scapular reference point (36′) from the nCOR (8′). The humeral reference point (34′), scapular reference point (36′) and pCOR (22′) as depicted in FIGS. 18 and 19 are illustrative only and do not necessarily reflect their exact direction or distance with respect to the nCOR (8′).

Referring now to FIGS. 20 through 24, shown are various views of one variation of a scapular baseplate (102′) in accordance with one possible embodiment. FIGS. 28 through 30 depict various views of another variation of a scapular baseplate (102″) in accordance with at least one further embodiment. In at least some embodiments, the scapular baseplate (102′, 102″) may optionally further comprise a vertical dimension (117′) (see FIGS. 22-24 and 29-30), a first side (118′) and a second side (122′) opposite of the first side (118′). Referring to FIGS. 22-24 and 29-30, the second side (122′) may optionally comprise a trunnion (124′) offset inferiorly to a center (127′) of the vertical dimension (117′). The trunnion (124′) may be adapted to engage with a glenosphere component (144′) (see engagement in FIG. 31). Referring to FIGS. 22, 23 and 30, the first side (118′) may optionally further comprise a mating surface (119′) with a scapular stem (120′). Referring to FIG. 25, the mating surface (119′) may optionally be adapted to approximately cooperate with a glenoid cavity (5′) of the scapula bone (6′). In at least some embodiments, the mating surface (119′) may be adapted to not extend below an inferior edge (13′) of the glenoid cavity (5′). The scapular stem (120′) may be adapted for insertion into, and rigid engagement with, the scapula bone (6′). In should be understood that the scapular stem (120′) may be of varying lengths, shapes and sizes. For example, the embodiment of the scapular baseplate (102′) in FIGS. 20 through 23 depicts a longer scapular stem (120′), whereas the embodiment of the scapular baseplate (102″) in FIGS. 28 and 30 depicts a shorter scapular stem (120′).

Referring to FIGS. 22 and 30, in at least some embodiments a thickness (154′) between the first side (118′) and the second side (122′) of the scapular baseplate (102′, 102″) may gradually increase along the vertical dimension (117′) from a bottom edge (158′) to a top edge (156′) of the scapular baseplate (102′, 102″). In some embodiments, such as the ones depicted throughout FIGS. 20 through 31A, the mating surface (119′) may optionally be curved. The curved mating surface (119′) may be adapted to prevent rotation of the scapular baseplate (102′) once engaged with a scapula bone (6′). Referring further to FIGS. 22, 25 and 30, in some embodiments having a mating surface (119′) that is curved, the curved mating surface (119′) of the first side (118′) of the scapular baseplate (102′, 102″) optionally forms an arc (160′) having a central axis (162′) extending in a direction that is angled downward with respect to the longitudinal axis (121′) of the scapular stem (120′) of the scapular baseplate (102′, 102″). In the present context, the “central axis” (162′) refers to an axis that extends from the center of arc (160′) and bisects arc (160″). In further embodiments having a mating surface (119′) that is curved, the curved mating surface (119′) describes a chord, and the chord may be at an angle (not shown) between 5 and 15 degrees from the second side (122′) of the scapular baseplate (102′, 102″). In some embodiments, the angle (not shown) between the second side (122′) of the scapular baseplate (102′, 102″) and the chord is equal to 10 degrees. In other embodiments, the curved mating surface (119′) may comprise a radius (not shown) of the arc (160′) that is optionally between 22 and 30 millimeters in length. Referring further to FIGS. 22 and 30, in further embodiments, the direction of the central axis (162′) of the curved mating surface (119′) may be at an angle (164′) between 10 and 25 degrees inferior to the longitudinal axis (121′) of the scapular stem (120′). In at least one embodiment, the direction of the central axis (162′) of the curved mating surface (119′) is at an angle (164′) 17 degrees inferior to the longitudinal axis (121′) of the scapular stem (120′).

Referring to FIGS. 22 and 30, in at least some embodiments, the scapular stem (120′) of the scapular baseplate (102′, 102″) may optionally extend outward, for example, at an angle between 1 and 20 degrees above the anatomical horizontal. Referring further to FIG. 22, in further embodiments, the trunnion (124′) comprises a longitudinal axis (129′) and the longitudinal axis (121′) of the scapular stem (120′) optionally extends outward at an angle (168′) between 3 and 15 degrees superior to the longitudinal axis (129′) of the trunnion (124′). In some embodiments, the longitudinal axis (121′) of the scapular stem (120′) is at an angle (168′) equal to 7 degrees superior to the longitudinal axis (129′) of the trunnion (124′). The angled scapular stem (120′) is adapted to prevent the scapular baseplate (102′, 102″), once implanted, from disengaging from the scapula bone (6′) when force is applied to the scapular baseplate (102′, 102″).

Referring to FIGS. 20, 24 and 28-29, in further embodiments, the second side (122′) of the scapular baseplate (102′, 102″) comprises a surface (123′) that may optionally comprise at least one recessed area (125′). In other embodiments, the surface (123′) of the second side (122′) may not have any recessed areas (125′). For example, the embodiment of FIG. 25 does not comprise a recessed area (125′). Referring to FIG. 24, in embodiments having a recessed area (125′), at least one recessed area (125′) may optionally be located superior to the center (127′) of the vertical dimension (117′). However, other embodiments with varying number of recessed areas at varying depths and locations are possible. For example, the scapular baseplate (102″) of FIGS. 28-29 comprises three recessed areas (125′) of varying depth, two of which extend beyond the center (127′) of the vertical dimension (117′). The at least one recessed area (125′) may be adapted to provide extended articulation of the humeral component (200′″) when rotated upwards. Referring again to FIGS. 20-22, 23-24 and 28-29, the scapular baseplate (102′, 102″) may optionally further comprise one or more holes (104′, 106′, 108′) extending from the second side (122′) to the first side (118′) of the scapular baseplate (102′, 102″). For example, the embodiment of FIGS. 28 and 29 comprises one such hole (104′), whereas the embodiment of FIGS. 20 through 24 comprises three holes (104′, 106′, 108′). However, other embodiments with varying number of holes are possible in accordance with the present scapular baseplate (102′, 102″). The one or more holes (104′, 106′, 108′) may each be adapted to receive a screw to engage and secure the scapular baseplate (102′, 102″) to a scapula bone (6′). Referring to FIG. 20, one or more holes (104′, 106′, 108′) may optionally each further comprise an internal thread (105′, 107′, 109′). Referring next to FIG. 23, the one or more holes (104′, 106′, 108′) may optionally each have an axis (113′) that is different than an axis (115′) of the internal thread (105′, 107′, 109′) of each of the respective one or more holes (104′, 106′, 108′). In embodiments with more than one hole, the holes (104′, 106′, 108′) may optionally have varying hole axes (113′) and/or varying internal thread axes (115′) with respect to one another. In at least one embodiment, the axis (115′) of the internal thread (105′, 107′, 109′) of the one or more holes (104′, 106′, 108′) may optionally be substantially normal to a surface (123′) of the second side (122′) of the scapular baseplate (102′, 102″). Referring to FIGS. 26 and 27, the one or more holes (104′, 106′, 108′) may each be adapted to receive a fastener. FIG. 26 shows an exploded view of a scapular baseplate (102′) and a screw (112′) with a locking cap (114′) adapted to be received one of the one or more holes (104′, 106′, 108′) and a trunnion screw (126′) with trunnion locking cap (128′) adapted to be received by the trunnion (124′) of the scapular baseplate (102′). FIG. 27 shows one of the one or more holes (106′) of the scapular baseplate (102′) having received the screw (112′) with locking cap (114′) and the trunnion (124′) having received trunnion screw (126′) with trunnion locking cap (128′). While screw (112′) is a depicted as a polyaxial screw, a single-axis screw could also be used. In embodiments where the hole axis (113′) is different than the internal thread axis (115′), screw (112′) may be inserted along the hole axis (113′) and the corresponding locking cap may be inserted along the internal thread axis (115′).

Referring next to FIGS. 31 through 34, the prosthetic joint assembly may optionally further comprise a glenosphere component (144′) adapted to be engaged by the trunnion (124′) of the scapular baseplate (102′, 102″). Referring further to FIG. 33, the glenosphere component (144′) may comprise a hollow glenosphere cover (132′) having an interior surface (133′) and an exterior surface (135′). In at least some embodiments, the glenosphere cover (132′) has a thickness between the interior surface (133′) and the exterior surface (135′) of at least 3 millimeters. Referring to FIG. 32, the glenosphere component (144′) may further comprise a solid glenosphere core (130′) having two sides (141′, 142′), a first side (141′) and a second side (142′). Referring back to FIG. 31, in at least some embodiments the exterior surface (135′) of the glenosphere cover (132′) may optionally comprise the convex surface (152′) of the second side (103′) of the scapular component (100′). Referring again to FIG. 34, the first side (141′) of the glenosphere core (130′) may optionally comprise an aperture (145′) adapted for rigid engagement with the trunnion (124′) of the scapular baseplate (102′, 102″) (see trunnion (124′) in FIGS. 20 and 28). Referring to FIG. 34, the glenosphere core (130′) may be adapted to assemble with the glenosphere cover (132′). In at least some embodiments, the glenosphere core (130′) is adapted to rigidly assemble with the glenosphere cover (132′) so as to prevent rotational motion between the glenosphere cover (132′) and the glenosphere core (130′). In other embodiments, when the glenosphere core (130′) and the glenosphere cover (132′) are assembled, the glenosphere cover (132′) may be adapted to freely rotate with respect to the glenosphere core (130′). In further embodiments, when the glenosphere cover (132′) and the glenosphere core (130′) are assembled, and the concave dish (236′) of the humeral component (200′″) engages the convex surface (152′) of the glenosphere cover (132′) (engagement not shown), the humeral component (200′″) freely swivels with respect to the glenosphere cover (132′) and the glenosphere core (130′) about the pCOR (22′) (see pCOR (22′) in FIG. 19). Referring further to FIGS. 32 through 34, in embodiments having a glenosphere component (144′), the glenosphere core (130′) may further comprise a hole (136′) extending from the aperture (145′) of the first side (141′) to the second side (142′) of the glenosphere core (130′). The glenosphere cover (132′) may optionally further comprise a hole (138′) extending from the interior surface (133′) to the exterior surface (135′) of the glenosphere cover (132′). The hole (136′) of the glenosphere core (130′) and the hole (138′) of the glenosphere cover (132′) may be adapted to align with one another and the trunnion (124′) of the scapular baseplate (102′, 102″) and may be adapted to receive a setting screw (not shown) to secure the glenosphere component (144′) to the scapular baseplate (102′, 102″) at the trunnion (124′) (as shown assembled in FIG. 31). Referring to FIGS. 26 and 27, the trunnion (124′) of the scapular baseplate (102′, 102″) may optionally comprise an internal thread adapted to engage an external thread of a trunnion locking cap (128′). The trunnion locking cap (128′) may be adapted to receive the setting screw (not shown) for engagement of the glenosphere component (144′) and the trunnion (124′).

A further object is to provide a method for prosthetically joining a humerus bone (4′) to a scapula bone (6′). The following is a listing of the generalized steps. It should be understood that the steps described herein may be carried out in any desired order, and additional steps may be added as desired and/or steps may be deleted as desired. In at least one embodiment, the method may comprise the step of providing a humeral component (200′″), such as the embodiment depicted in FIG. 35. In some embodiments, the humeral component (200′″) may have two opposite ends, the first end (201′) optionally comprising a humeral stem (202′) adapted for rigid engagement with the medullar canal (9′) of the humerus bone (4′) (see FIG. 18) and the second end (203′) optionally comprising a concave dish (236′). The humeral stem (202′) may optionally comprise a single, unitary component.

The method may further comprise the step of providing a scapular component (100′), such as the embodiment depicted in FIG. 31, having two opposite sides (101′, 103′). In some embodiments, the first side (101′) of the scapular component (100′) may optionally comprise a scapular baseplate (102′, 102″), such as the one depicted in FIG. 20-24 or 28-30, adapted for rigid engagement with the scapula bone (6′) at the glenoid cavity (5′) (see FIG. 25). The second side (103′) may optionally comprise a convex surface (152′) adapted to engage the concave dish (236′) of the humeral component (200″).

The method may next comprise the step of relocating the humerus bone (4′) with respect to the scapula bone (6′). FIG. 18 illustrates a humerus bone (4′) and scapula bone (6′) before relocating the humerus bone (4′). FIG. 19 illustrates the humerus bone (4′) relocated with respect to the scapula bone (6′) in accordance with one possible embodiment. Referring to FIG. 18, the step of relocating the humerus bone (4′) may comprise establishing a humeral reference point (34′) in a direction that is medial to the nCOR (8′) and a distance from the nCOR (8′) approximately equal to between 60% and 80%, and optionally 70%, of the humeral head radius distance (3′). In at least one embodiment, the humeral reference point (34′) may be established in a direction 30 to 60 degrees above horizontal and medial to the nCOR (8′). Referring again to FIG. 18, relocating the humerus bone (4′) may further comprise establishing a scapular reference point (36′) that is in a direction from the nCOR that is approximately normal (i.e., within 15 degrees of normal) and medial to the direction of the humeral reference point (34′), and at a distance from the nCOR (8′) approximately equal to between 60% and 80%, and optionally 70%, of the humeral head radius distance (3′). Referring next to FIG. 19, relocating the humerus bone (4′) may next comprise repositioning the humerus bone (4′) so that the humeral reference point (34′) is superimposed on the scapular reference point (36′) and establishing a prosthetic center of rotation (pCOR) (22′) at the scapular reference point (36′). In further embodiments, the distance of the humeral reference point (34′) from the nCOR (8′) may be approximately equal to the distance of the scapular reference point (36′) from the nCOR (8′). As discussed previously, the humeral reference point (34′), scapular reference point (36′) and pCOR (22′) as depicted in FIGS. 18 and 19 are illustrative only and do not necessarily reflect their exact direction or distance with respect to the nCOR (8′).

The method may further comprise the step of resecting the humeral head (2′) of the humerus bone (4′) and engaging the humeral stem (202′, 202″) (see FIGS. 35-38) of the humeral component (200′″) to the medullar canal (9′) of the repositioned humerus bone (4′) (see repositioned humerus bone (4′) in FIG. 19). In some embodiments, engaging the humeral stem (202′) to the medullar canal (9′) of the repositioned humerus bone (4′) may optionally further comprise the step of inserting a screw through the humerus bone (4′) and into a hole (248′) located at a bottom end (246′) of the humeral stem (202′) (see FIG. 35), the hole (248′) being transverse to a longitudinal axis (211′) of the humeral stem (202′). Referring to FIG. 25, the method may next comprise engaging the scapular baseplate (102′, 102″) (see other optional embodiments in FIGS. 20-24 and 28-30) to the scapula bone (6′). Next, the method may comprise the step of engaging the concave dish (236′) of the humeral component (200′″) (see, for example, FIG. 35) with the convex surface (152′) of the scapular component (100′) (see, for example, FIG. 31) so that the relocated humerus bone (4′) freely swivels with respect to the scapula bone (6′) about the pCOR (22′) located at the scapular reference point (36′) (see FIG. 19).

As described above, the scapular baseplate (102′, 102″) (see, for example FIGS. 20-24 and FIGS. 28-30) of the scapular component (100′) may optionally further comprise a vertical dimension (117′); a first side (118′) comprising a mating surface (119′) with a scapular stem (120′), the mating surface (119′) adapted to approximately cooperate with a glenoid cavity (5′) of the scapula bone (6′), the scapular stem (120′) adapted for insertion into, and rigid engagement with, the scapula bone (6′); and a second side (122′) opposite of the first side (118′) comprising a trunnion (124′) offset inferiorly relative to a center (127′) of the vertical dimension (117′). In such embodiments, the step of engaging the scapular baseplate (102′, 102″) to the scapula bone (6′) may further comprise engaging the scapular stem (120′) of the scapular component (100′) to the scapula bone (6′) such that the mating surface (119′) cooperates with, and does not extend below an inferior edge (13′) of, the glenoid cavity (5′) of the scapula bone (6′) (see, for example, FIG. 25).

Referring again to FIGS. 22 and 30, a thickness (154′) between the first side (118′) and the second side (122′) of the scapular baseplate (102′, 102″) may gradually increase along the vertical dimension (117′) from a bottom edge (158′) to a top edge (156′) of the scapular baseplate (102′, 102″). In some embodiments, such as the ones depicted throughout FIGS. 20 through 31A, the mating surface (119′) may optionally be curved. Referring to FIGS. 22, 25 and 30, and as described above, in embodiments having a mating surface (119′) that is curved, the curved mating surface (119′) of the first side (118′) of the scapular baseplate (102′, 102″) optionally forms an arc (160′) having a central axis (162′) extending in a direction that is angled downward with respect to a longitudinal axis of the scapular stem (120′) of the scapular baseplate (102′, 102″). The curved mating surface (119′) may comprise a radius (not shown) of the arc (160′) between 22 and 30 millimeters in length. The direction of the central axis (162′) of the curved mating surface (119′) may be at an angle (164′) between 10 and 25 degrees inferior to the longitudinal axis (121′) of the scapular stem (120′). In at least one embodiment, the direction of the central axis (162′) of the curved mating surface (119′) arc (160′) is at an angle (164′) 17 degrees inferior to the longitudinal axis (121′) of the scapular stem (120′).

Referring again to FIGS. 31 and 31A, in some embodiments, the convex surface (152′) of the scapular component (100′) comprises a central axis (153′) and the longitudinal axis (121′) of the scapular stem (120′) of the scapular baseplate (102′, 102″) optionally extends outward, for example, at an angle (166′) between 3 and 15 degrees superior to the central axis (153′) of the convex surface (152′). In some embodiments, the scapular stem (120′) is at an angle (166′) equal to 7 degrees superior to the central axis (153′) of the convex surface (152′). Referring to FIG. 24, the second side (122′) of the scapular baseplate (102′, 102″) may comprise a surface (123′) having one or more recessed areas (125′) that are optionally located superior to the center (127′) of the vertical dimension (117′). The recessed area (125′) may be adapted to increase the range of motion and reduce impingement of the humeral component (200′″) when the relocated humerus bone (4′) swivels upward with respect to the scapula bone (6′) about the pCOR (22′).

Referring to FIGS. 31 through 34, the method may optionally further comprise the step of providing a glenosphere component (144′) having a hollow glenosphere cover (132′) and a solid glenosphere core (130′). The glenosphere cover (132′) may have the convex surface (152′) of the scapular component (100′). The glenosphere core (130′) may have a first side (141′) with an aperture (145′) adapted for rigid engagement with the trunnion (126′) of the scapular baseplate (102′, 102″) and a second side (142′) adapted to assemble with the glenosphere cover (132′). In some embodiments, the second side (142′) of the scapular baseplate (102′, 102″) may be adapted to rigidly assemble with the glenosphere cover (132′). In such embodiments having a glenosphere component (144′), the method may further comprise engaging the aperture (145′) of the glenosphere core (130′) with the trunnion (124′) of the scapular baseplate (102′, 102″) and assembling the glenosphere core (130′) with the glenosphere cover (132′) before engaging the concave dish (236′) of the humeral component (200′″) with the convex surface (152′) of the scapular component (100′). In at least some embodiments, after assembling the glenosphere core (130′) with the glenosphere cover (132′), the method may further comprise the step of securing the glenosphere component (144′) to the scapular baseplate (102′, 102″) by inserting a setting screw into a hole (138′) of the glenosphere cover (132′) through to a corresponding hole (136′) of the glenosphere core (130′) and into the trunnion (124′) of the scapular baseplate (102′, 102″), the holes (136′, 138′) of the glenosphere core (130′) and glenosphere cover (132′) adapted to align with one another and the trunnion (124′). The trunnion (124′) optionally comprises an internal thread adapted to engage an external thread of a trunnion locking cap (128′), and the trunnion locking cap (128′) may be adapted to receive the setting screw for engagement of the glenosphere component (144′) and the trunnion (124′).

In at least some embodiments, the step of engaging the scapular baseplate (102′, 102″) to the scapula bone (6′) may optionally further comprise inserting one or more screws through a respective one or more holes (104′, 106′, 108′) (see FIG. 20) of the scapular baseplate (102′, 102″) and into the scapula bone (6′), the one or more holes (104′, 106′, 108′) extending from the second side (122′) to the first side (118′) of the scapular baseplate (102′, 102″). The step of inserting the one or more screws though respective holes (104′, 106′, 108′) may optionally further comprise inserting the one or more screws along an axis (113′) of the hole (104′, 106′, 108′) that is different than an axis (115′) of an internal thread (105′, 107′, 109′) (see FIG. 20) of the respective one or more holes (104′, 106′, 108′) (see FIG. 23). Referring further to FIG. 23, in some embodiments, the axis (115′) of the internal thread of the one or more holes (104′, 106′, 108′) may be substantially normal to the surface (123′) of the second side (122′) of the scapular baseplate (102′, 102″).

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from any of the following materials: (a) any biocompatible material (which biocompatible material may be treated to permit surface bone ingrowth or prohibit surface bone ingrowth-depending upon the desire of the surgeon); (b) a plastic; (c) a fiber; (d) a polymer; (e) a metal (a pure metal and/or an alloy); (f) any combination thereof. Further still, any number of protrusions (e.g., such as for initial fixation by forming a bond with cement and/or such as for supplemental fixation by forming a bond with cement) may be utilized with a given prosthesis. Further still, any number of female features that increase the bonding area may be utilized with a given prosthesis. Further still, any number of male features that could dig into the bone so that initial/supplemental fixation can be improved may be utilized with a given prosthesis. Further still, any number of bone screws (e.g., such as for initial fixation and/or such as for supplemental fixation) may be utilized with a given prosthesis. Further still, any steps described herein may be carried out in any desired order (and any additional steps may be added as desired and/or any steps may be deleted as desired).

As used herein, the terms “substantial” and “substantially” refer to the complete or nearly complete extent or degrees of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context, which one of ordinary skill in the art would be familiar with. However, generally speaking the nearness of completion will be so as to have the same overall result as if absolute or total completion were obtained.

The use of term “substantial” or “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

In addition, various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method for prosthetically joining a humerus bone to a scapula bone, the humerus bone having a medullar canal, a humeral head, and a humeral head radius distance, the humerus bone having a natural center of rotation (nCOR) with respect to the scapula bone, the method comprising the steps of:
providing a humeral component having two opposite ends, the first end comprising a humeral stem adapted for rigid engagement with the medullar canal of the humerus bone and the second end comprising a concave dish;
providing a scapular component having two opposite sides, the first side comprising a scapular baseplate adapted for rigid engagement with the scapula bone, the second side comprising a convex surface adapted to engage the concave dish;
relocating the humerus bone with respect to the scapula bone by (a) establishing a humeral reference point in a direction that is 30 to 60 degrees above horizontal and medial to the nCOR and a distance from the nCOR approximately equal to between 60% and 80% of the humeral head radius distance; (b) establishing a scapular reference point that is in a direction from the nCOR that is approximately normal and medial to the direction of the humeral reference point, and at a distance from the nCOR approximately equal to between 60% and 80% of the humeral head radius distance; (c) repositioning the humerus bone so that the humeral reference point is superimposed on the scapular reference point and establishing a prosthetic center of rotation (pCOR) at the scapular reference point;
resecting the humeral head and engaging the humeral stem of the humeral component to the medullar canal of the repositioned humerus bone;
engaging the scapular baseplate to the scapula bone; and
engaging the concave dish of the humeral component with the convex surface of the scapular component so that the relocated humerus bone freely swivels with respect to the scapula bone about the pCOR.

2. The method of claim 1 wherein the convex surface comprises a central axis and the scapular baseplate comprises a scapular stem having a longitudinal axis that extends outward at an angle between 3 and 15 degrees superior to the central axis of the convex surface.

3. The method of claim 1 wherein the scapular baseplate of the scapular component further comprises:
a vertical dimension;
a first side comprising a mating surface with a scapular stem, the mating surface adapted to cooperate with a glenoid cavity of the scapula bone, the scapular stem adapted for insertion into, and rigid engagement with, the scapula bone; and
a second side opposite of the first side comprising a trunnion offset inferiorly relative to a center of the vertical dimension.

4. The method of claim 3 wherein engaging the scapular baseplate to the scapula bone further comprises engaging the scapular stem of the scapular component to the scapula bone such that the mating surface cooperates with, and does not extend below an inferior edge of, the glenoid cavity of the scapula bone.

5. The method of claim 3 wherein a thickness between the first side and the second side of the scapular baseplate gradually increases along the vertical dimension from a bottom edge to a top edge of the scapular baseplate.

6. The method of claim 3 wherein the second side of the scapular baseplate further comprises a surface having a recessed area located superior to the center of the vertical dimension, the recessed area adapted to increase range of motion and reduce impingement of the humeral component when the relocated humerus bone swivels upward with respect to the scapula bone about the pCOR.

7. The method of claim 3 wherein the mating surface is curved.

8. The method of claim 7 wherein the curved mating surface comprises a radius that is between 22 and 30 millimeters in length.

9. The method of claim 7 wherein the curved mating surface of the scapular baseplate comprises a central axis extending in a direction that is angled downward with respect to a longitudinal axis of the scapular stem of the scapular baseplate.

10. The method of claim 9 wherein the direction of the central axis of the curved mating surface is between 10 and 25 degrees inferior to the longitudinal axis of the scapular stem of the scapular baseplate.

11. The method of claim 3 further comprising, providing a glenosphere component having a hollow glenosphere cover and a solid glenosphere core, the glenosphere cover comprising the convex surface of the scapular component, the glenosphere core comprising a first side with an aperture adapted for rigid engagement with the trunnion of the scapular baseplate and a second side adapted to assemble with the glenosphere cover.

12. The method of claim 11 further comprising, engaging the aperture of the glenosphere core with the trunnion of the scapular baseplate and assembling the glenosphere core with the glenosphere cover before engaging the concave dish of the humeral component with the convex surface of the scapular component.

13. The method of claim 12 further comprising, securing the glenosphere component to the scapular baseplate after assembling the glenosphere core with the glenosphere cover by inserting a setting screw into a hole of the glenosphere cover through to a corresponding hole of the glenosphere core and into the trunnion of the scapular baseplate, the holes of the glenosphere core and the glenosphere cover adapted to align with one another and the trunnion.

14. The method of claim 13 wherein the trunnion comprises an internal thread adapted to engage an external thread of a locking cap, the locking cap adapted to receive the setting screw for engagement of the glenosphere component and the trunnion.

15. The method of claim 3 wherein engaging the scapular baseplate to the scapula bone further comprises inserting one or more screws through a respective one or more holes of the scapular baseplate and into the scapula bone, the one or more holes extending from the second side to the first side of the scapular baseplate.

16. The method of claim 15 wherein inserting the one or more screws through the respective one or more holes of the scapular baseplate further comprises inserting the one or more screws along an axis of the hole that is different than an axis of an internal thread of the respective one or more holes.

17. The method of claim 16 wherein the axis of the internal thread of the one or more holes is substantially normal to a surface of the second side of the scapular baseplate.

18. The method of claim 1 wherein the humeral stem of the humeral component comprises a single, unitary component.

19. The method of claim 1 wherein engaging the humeral stem of the humeral component to the medullar canal of the repositioned humerus bone further comprises inserting a screw through the humerus bone and into a hole located at a bottom end of the humeral stem, the hole transverse to a longitudinal axis of the humeral stem.

20. A prosthetic joint assembly for joining a humerus bone to a scapula bone, the humerus bone having a humeral head radius distance, the humerus bone having a natural center of rotation (nCOR) with respect to the scapula bone, the prosthetic joint assembly comprising:
a humeral component having two opposite ends, the first end comprising a humeral stem adapted for rigid engagement with the humerus bone and the second end comprising a concave dish; and
a scapular component having two opposite sides, the first side comprising a scapular baseplate adapted for rigid engagement with the scapula bone, the second side comprising a convex surface adapted to engage the concave dish;
wherein the geometry of the humeral component and the scapular component is such that when the humeral stem is engaged with the humerus bone, the scapular baseplate is engaged with the scapula bone, and the concave dish and the convex surface are engaged, the humerus bone freely swivels with respect to the scapular component about a prosthetic center of rotation (pCOR), and the humerus bone is shifted to a relocated position with respect to the scapula bone; and
wherein the geometry of the humeral component and the scapular component is such that the relocated position of the humerus bone with respect to the scapula bone is fixed by (a) calculating a humeral reference point in a direction that is 30 to 60 degrees above horizontal and medial to the nCOR and a distance from the nCOR approximately equal to between 60% and 80% the humeral head radius distance; (b) calculating a scapular reference point that is in a direction from the nCOR that is approximately normal and medial to the direction of the humeral reference point and at a distance from the nCOR approximately equal to between 60% and 80% the humeral head radius distance; (c) moving the humerus bone so that the humeral reference point is superimposed on the scapular reference point and establishing the pCOR at the scapular reference point.

21. The prosthetic joint assembly of claim 20 wherein the convex surface comprises a central axis and the scapular baseplate comprises a scapular stem having a longitudinal axis that extends outward at an angle between 3 and 15 degrees superior to the central axis of the convex surface.

22. The prosthetic joint assembly of claim 20 wherein the scapular baseplate further comprises:
a vertical dimension;
a first side comprising a mating surface with a scapular stem, the mating surface adapted to cooperate with a glenoid cavity of the scapula bone, the scapular stem adapted for insertion into, and rigid engagement with, the scapula bone; and
a second side opposite of the first side comprising a trunnion offset inferiorly relative to a center of the vertical dimension.

23. The prosthetic joint assembly of claim 22 wherein a thickness between the first side and the second side of the scapular baseplate gradually increases along the vertical dimension from a bottom edge to a top edge of the scapular baseplate.

24. The prosthetic joint assembly of claim 22 wherein the second side of the scapular baseplate further comprises a surface, the surface comprising a recessed area located superior to the center of the vertical dimension, the recessed area adapted to provide extended articulation of the humeral component when rotated upwards.

25. The prosthetic joint assembly of claim 22 wherein the mating surface is curved.

26. The prosthetic joint assembly of claim 25 wherein the curved mating surface comprises a radius that is between 22 and 30 millimeters in length.

27. The prosthetic joint assembly of claim 25 wherein the curved mating surface of the scapular baseplate comprises a central axis extending in a direction that is angled downward with respect to a longitudinal axis of the scapular stem of the scapular baseplate.

28. The prosthetic joint assembly of claim 27 wherein the direction of the central axis of the curved mating surface is between 10 and 25 degrees inferior to the longitudinal axis of the scapular stem.

29. The prosthetic joint assembly of claim 22 further comprising a glenosphere component adapted to be engaged by the trunnion of the scapular baseplate, the glenosphere component comprising:
a hollow glenosphere cover having exterior and interior surfaces, the exterior surface comprising the convex surface of the scapular component; and
a solid glenosphere core having two sides, the first side comprising an aperture adapted for rigid engagement with the trunnion of the scapular baseplate, the glenosphere core adapted to assemble with the glenosphere cover;
wherein when the glenosphere cover and the glenosphere core are assembled, and the concave dish of the humeral component engages the convex surface of the glenosphere cover, the humeral component freely swivels with respect to the glenosphere cover and the glenosphere core about the pCOR.

30. The prosthetic joint assembly of claim 29 wherein the glenosphere core is adapted to rigidly assemble with the glenosphere cover so as to prevent rotational motion between the glenosphere cover and the glenosphere core.

31. The prosthetic joint assembly of claim 29 wherein:
the glenosphere core further comprises a hole extending from the aperture of the first side to the second side;
the glenosphere cover further comprises a hole extending from the interior surface to the exterior surface;
wherein the hole of the glenosphere core and the hole of the glenosphere cover are adapted to align with one another and the trunnion of the scapular baseplate; and
wherein the hole of the glenosphere core and the hole of the glenosphere cover are adapted to receive a setting screw to secure the glenosphere component to the scapular baseplate at the trunnion, the trunnion comprising an internal thread adapted to engage an external thread of a locking cap, the locking cap adapted to receive the setting screw for engagement of the glenosphere component and the trunnion.

32. The prosthetic joint assembly of claim 22 wherein the scapular baseplate further comprises one or more holes extending from the second side to the first side of the scapular baseplate.

33. The prosthetic joint assembly of claim 32 wherein the one or more holes each further comprise an internal thread, the one or more holes each having an axis that is different than an axis of the internal thread of each of the respective one or more holes.

34. The prosthetic joint assembly of claim 33 wherein the axis of the internal thread of the one or more holes is substantially normal to a surface of the second side of the scapular baseplate.

35. The prosthetic joint assembly of claim 20 wherein the humeral stem comprises a single, unitary component.

36. The prosthetic joint assembly of claim 20 wherein a bottom end of the humeral stem comprises a hole extending through the humeral stem transverse to a longitudinal axis of the humeral stem, the hole adapted to receive a screw to rigidly engage the humeral component to the humerus bone.

37. A baseplate for engagement with a scapula bone, the scapula bone having a glenoid cavity, the baseplate comprising:
a vertical dimension;
a first side comprising a mating surface with a scapular stem, the mating surface adapted to cooperate with the glenoid cavity, the scapular stem adapted for insertion into, and rigid engagement with, the scapula bone; and
a second side opposite of the first side comprising a trunnion offset inferiorly relative to a center of the vertical dimension, the trunnion being adapted to engage with a glenosphere component.

38. The baseplate of claim 37 wherein the trunnion comprises a longitudinal axis and the scapular stem comprises a longitudinal axis that extends outward at an angle between 3 and 15 degrees superior to the longitudinal axis of the trunnion.

39. The baseplate of claim 37 wherein a thickness between the first side and the second side gradually increases along the vertical dimension from a bottom edge to a top edge of the baseplate.

40. The baseplate of claim 37 wherein the second side further comprises a surface, the surface comprising a recessed area located superior to the center of the vertical dimension.

41. The baseplate of claim 37 wherein the mating surface is curved.

42. The baseplate of claim 41 wherein the curved mating surface comprises a radius that is between 22 and 30 millimeters in length.

43. The baseplate of claim 41 wherein the curved mating surface comprises a central axis extending in a direction that is angled downward with respect to a longitudinal axis of the scapular stem.

44. The baseplate of claim 43 wherein the direction of the central axis of the curved mating surface is between 10 and 25 degrees inferior to the longitudinal axis of the scapular stem.

45. The baseplate of claim 37 further comprising one or more holes extending from the second side to the first side.

46. The baseplate of claim 45 wherein the one or more holes each comprise an internal thread, the one or more holes each having an axis that is different than an axis of the internal thread of each of the respective one or more holes.

47. The baseplate of claim 46 wherein the axis of the internal thread is substantially normal to a surface of the second side of the baseplate.
